(19) United States
(12) Patent Application Publication
(10) Pub. No.: US 2024/0076272 A1
(43) Pub. Date: Mar. 7, 2024

(54) Activators of Heme Regulated Inhibitor Kinase (HRI)

(71) Applicant: The Brigham and Women`s Hospital, Inc., Boston, MA (US)

(72) Inventors: Bertal Huseyin Aktas, Newton, MA (US); Michael Chorev, Chestnut Hill, MA (US); Qingwen Zhang, Boston, MA (US)

(21) Appl. No.: 17/766,945

(22) PCT Filed: Oct. 8, 2020
(86) PCT No.: PCT/US2020/054712

§ 371 Date: Apr. 6, 2022

Related U.S. Application Data
(60) Provisional application No. 62/912,108, filed on Oct. 8, 2019.

(51) Int. Cl. C07D 239/47; A61P 35/00; C07C 275/36

(52) U.S. Cl. CPC C07D 239/47; A61P 35/00; C07C 275/36; C07C 2601/04; C07C 2601/08; C07C 2601/14

(57) ABSTRACT

The present application provides compounds that modulate the activity of one or more eIF2α kinases. Pharmaceutical compositions and methods of treating diseases related to one or more eIF2α kinases are also provided.

### CROSS-REFERENCE TO RELATED APPLICATIONS

This application claims the benefit of U.S. Provisional Application Ser. No. 62/912,108, filed Oct. 8, 2019, the disclosure of which is incorporated herein by reference in its entirety.

### FEDERALLY SPONSORED RESEARCH OR DEVELOPMENT

This invention was made with Government support under Grant No. 1RO1CA152312 awarded by the National Institutes of Health. The Government has certain rights in the invention.

### TECHNICAL FIELD

The present application provides compounds that modulate the activity of one or more eIF2α kinases and are useful in the treatment of diseases related to one or more eIF2α kinases.

### BACKGROUND

Heme-regulated inhibitor (HRI), a eukaryotic translation initiation factor 2 alpha (eIF2α) kinase, is important for coupling protein synthesis to heme availability in reticulocytes and adaptation to various environmental stressors in all cells. HRI modifies the severity of several hemoglobin misfolding disorders including β-thalassemia. Small molecule activators of HRI are useful for studying normal- and patho-biology of this kinase as well as for the treatment of various human disorders for which activation of HRI or phosphorylation of eIF2α may be beneficial.

### SUMMARY

The present application provides, inter alia, a compound of Formula I:

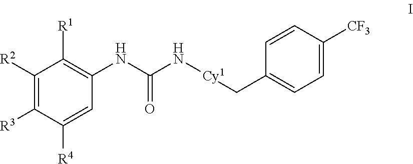

or a pharmaceutically acceptable salt thereof, wherein:

Cy¹ is selected from the group consisting of a C₃₋₁₀ cycloalkyl ring and a 5-10 membered heteroaryl ring;

R¹, R², R³, and R⁴ are each independently selected from the group consisting of H, halo, C₁₋₆ alkyl, C₁₋₆ haloalkyl, and cyano, wherein at least one of R¹, R², R³, and R⁴ is not H;

provided that the compound of Formula I is not selected from the group consisting of:

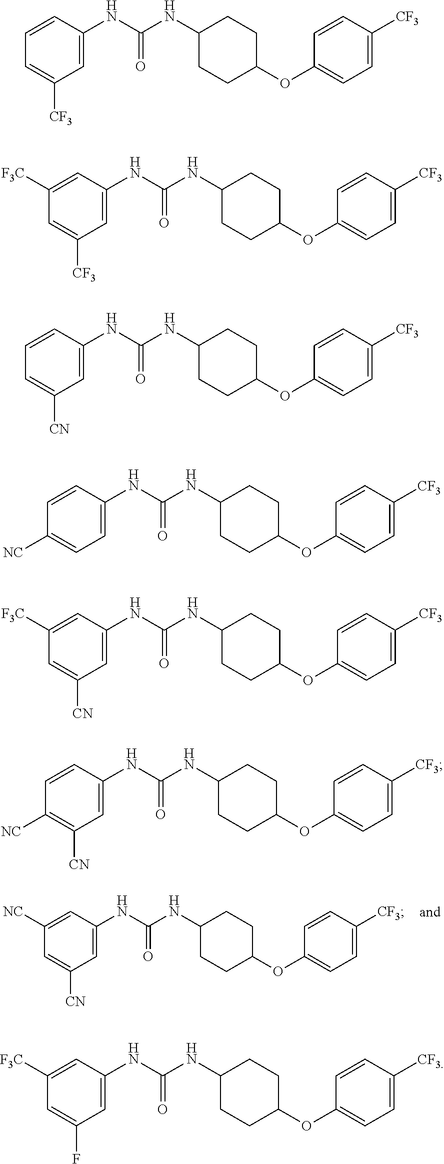

In some embodiments, Cy¹ is a C₃₋₁₀ cycloalkyl ring. In some embodiments, Cy¹ is a C₃₋₆ cycloalkyl ring. In some embodiments, Cy¹ is selected from the group consisting of cyclobut-1,3-diyl, cyclopent-1,3-diyl, and cyclohex-1,4-diyl. In some embodiments, Cy¹ is a 5-10 membered heteroaryl ring. In some embodiments, Cy¹ is a 5-6 membered heteroaryl ring. In some embodiments, Cy¹ is pyrimidinyl.

In some embodiments, R¹ is selected from the group consisting of H, halo, and C₁₋₃ alkyl. In some embodiments, R¹ is selected from the group consisting of H, fluoro, and methyl.

In some embodiments, R² is selected from the group consisting of H, halo, C₁₋₃ alkyl, C₁₋₃ haloalkyl, and cyano. In some embodiments, R² is selected from the group consisting of H, fluoro, methyl, trifluoromethyl, and cyano.

In some embodiments, R³ is selected from the group consisting of H, halo, and cyano. In some embodiments, R³ is selected from the group consisting of H, fluoro, and cyano.

In some embodiments, R⁴ is selected from the group consisting of H, halo, and cyano. In some embodiments, R⁴ is selected from the group consisting of H, fluoro, and cyano.

In some embodiments:

R¹ is selected from the group consisting of H, halo, and C₁₋₆ alkyl;

R² is selected from the group consisting of H, halo, C₁₋₆ alkyl, C₁₋₆ haloalkyl, and cyano;

R³ is selected from the group consisting of H, halo, and cyano; and

R⁴ is selected from the group consisting of H, halo, and cyano.

In some embodiments: R¹ is selected from the group consisting of H, halo, and C₁₋₃ alkyl;

R² is selected from the group consisting of H, halo, C₁₋₃ alkyl, C₁₋₃ haloalkyl, and cyano;

R³ is selected from the group consisting of H, halo, and cyano; and
R⁴ is selected from the group consisting of H, halo, and cyano.

In some embodiments: R¹ is selected from the group consisting of H, fluoro, and C₁₋₃ alkyl;

R² is selected from the group consisting of H, fluoro, C₁₋₃ alkyl, C₁₋₃ haloalkyl, and cyano;

R³ is selected from the group consisting of H, fluoro, and cyano; and

R⁴ is selected from the group consisting of H, fluoro, and cyano.

In some embodiments, two of R¹, R², R³, and R⁴ are H.

In some embodiments, three of R¹, R², R³, and R⁴ are H.

In some embodiments, the compound of Formula I is a compound of Formula II:

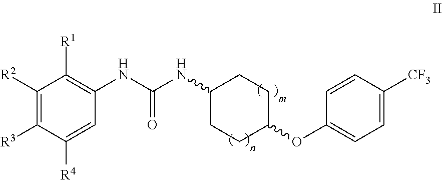

wherein n and m are each independently 0, 1, 2, or 3.

In some embodiments, the compound of Formula I is a compound of Formula III:

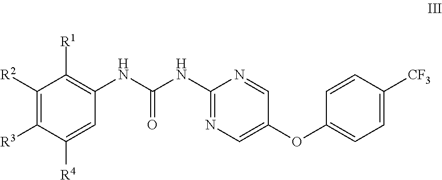

or a pharmaceutically acceptable salt thereof.

In some embodiments, the compound provided herein is selected from the group consisting of:

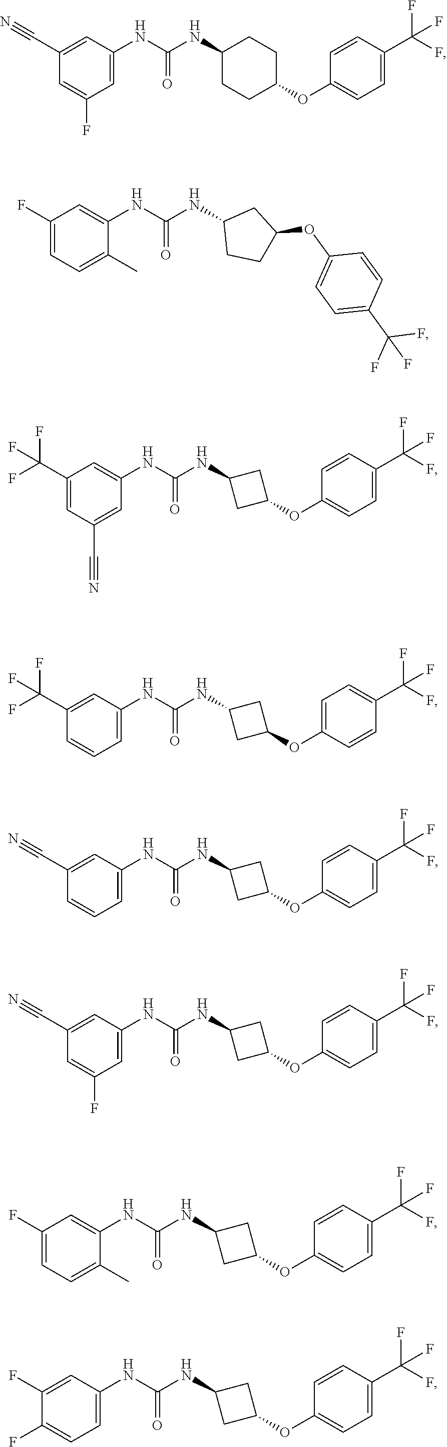

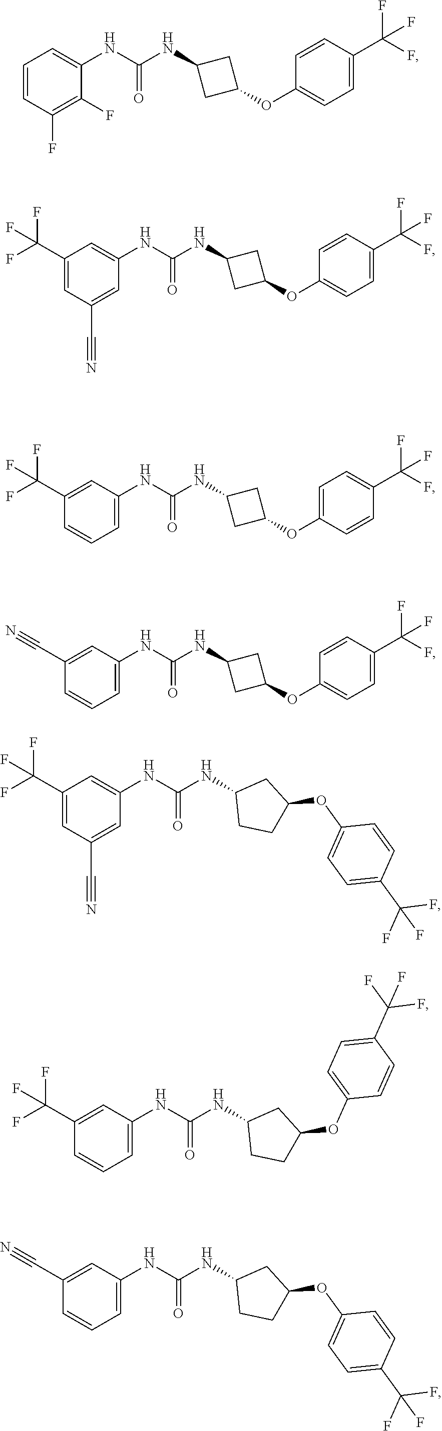

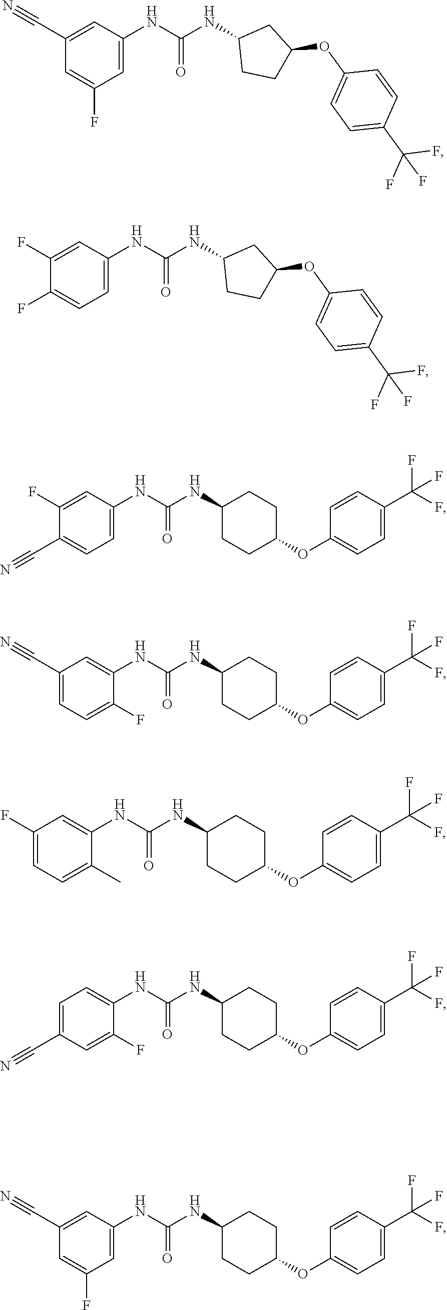

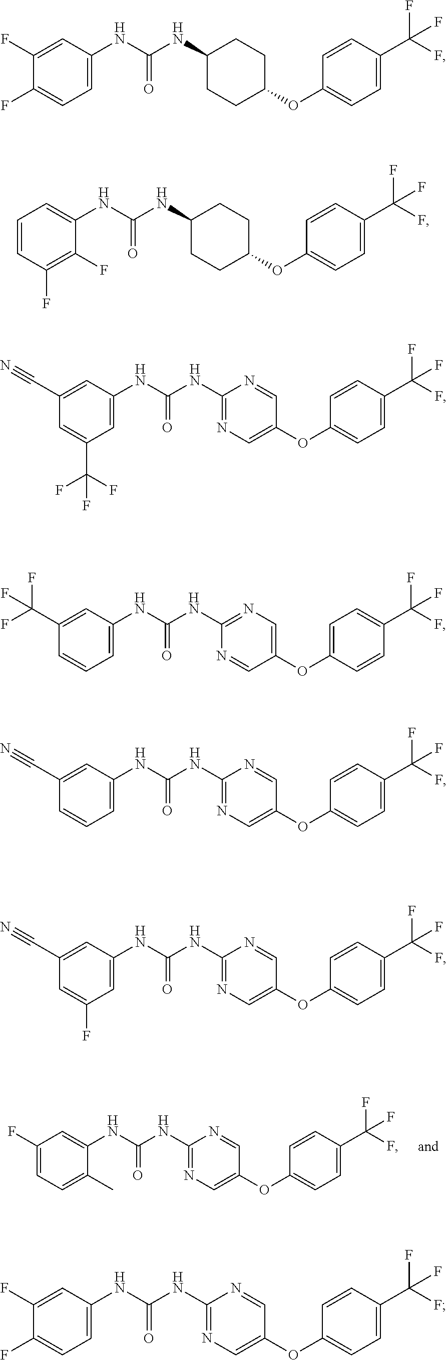

or a pharmaceutically acceptable salt thereof.

The present application further provides a pharmaceutical composition comprising a compound provided herein, or a pharmaceutically acceptable salt thereof, and a pharmaceutically acceptable excipient.

The present application further provides a method of activating one or more eIF2α kinases in a cell, the method comprising contacting the cell with an effective amount of a compound provided herein, or a pharmaceutically acceptable salt thereof.

The present application further provides a method of treating a disease or disorder associated with abnormal activity or expression of one or more eIF2α kinases in a patient, the method comprising administering to the patient a therapeutically effective amount of a compound provided herein, or a pharmaceutically acceptable salt thereof.

In some embodiments, the disease or disorder is cancer. In some embodiments, the cancer is selected from the group consisting of cervical cancer, liver cancer, bile duct cancer, eye cancer, esophageal cancer, head and neck cancer, brain cancer, prostate cancer, pancreatic cancer, skin cancer, testicular cancer, breast cancer, uterine cancer, penile cancer, small intestine cancer, colon cancer, stomach cancer, bladder cancer, anal cancer, lung cancer, lymphoma, leukemia, thyroid cancer, bone cancer, kidney cancer, ovarian cancer, and multiple myeloma. In some embodiments, the cancer is selected from the group consisting of breast cancer and skin cancer.

In some embodiments, the disease or disorder is hemolytic anemia not caused by an infectious agent. In some embodiments, the hemolytic anemia is selected from the group consisting of erythropoietic protoporphyria, α-thalassemia, β-thalassemia, δ-thalassemia, sideroblastic anemia, unstable hemoglobin hemolytic anemia, and iron-deficiency anemia. In some embodiments, the hemolytic anemia is β-thalassemia.

In some embodiments, the disease or disorder is Wolcott-Rallison syndrome.

In some embodiments, the disease or disorder is a neurodegenerative or motor neuron disease. In some embodiments, the neurodegenerative or motor neuron disease is selected from the group consisting of amyotrophic lateral sclerosis, Alzheimer's disease, Parkinson's disease, and Huntington's disease. In some embodiments, the neurodegenerative disease is Alzheimer's disease.

In some embodiments, the disease or disorder is selected from the group consisting of diabetes, non-alcoholic fatty liver disease, and tuberous sclerosis complex.

In some embodiments, the disease or disorder is an autism spectrum disorder. In some embodiments, the autism spectrum disorder is selected from the group consisting of Asperger's syndrome, autistic disorder, Rett syndrome, childhood disintegrative disorder, and pervasive developmental disorder, not otherwise specified (PDD-NOS).

In some embodiments, the disease or disorder is a ribosomal defect disease. In some embodiments, the ribosomal defect disease is selected from the group consisting of Shwachman-Bodian-Diamond syndrome, Diamond Blackfan anemia, and cartilage hair hypoplasia.

In some embodiments, a mental retardation disorder. In some embodiments, the mental retardation disorder is fragile-X syndrome.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 shows an X-ray ORTEP diagram of compound A-V.

FIGS. 2A-2B show HRI activators of the present application induce eIF2α phosphorylation and modify its downstream effectors. In FIG. 2A, CRL-2813 cells were treated with 5 μM of each compound for 2 hours, cell lysates were probed with phosphorylated (P-eIF2α) and total eIF2α (T-eIF2α) and β-actin specific antibodies. In FIG. 2B, CRL-2813 cells were treated with 5 μM HRI activators for 8 hours and lysates were immunoblotted with antibodies against CHOP, p27^{KiPl}, cyclin D1 and β-actin.

FIG. 3 shows that inhibition of cell proliferation by compounds of the present application is dependent on HRI. MCF-7 cells transfected with (solid bars) or without (hatched bars) siRNA targeting HRI were treated with the indicated concentrations of each compound for three days starting one day after transfection. Cell proliferation was measured by sulforhodamine B assay as previously reported (see e.g., Chen et al, Nat. Chem. Biol. 7 (2011) 610-616).

FIG. 4 shows a hypothetical model of HRI activation by 4-CF₃-ΦOcHΦUs. HRI binding of heme or cellular chaperones such as HSP-90 and/or other endogenous inhibitors cause the N-terminal domain (NTD) to interact with kinase domain such that kinase domain is inactivated and kept from binding the eIF2α.

### DETAILED DESCRIPTION

Previous reports have described 1-((1,4-trans)-4-aryloxycyclohexyl)-3-arylureas (cHAUs) as specific HRI activators and demonstrated their potential as molecular probes for studying HRI biology and as compounds for treatment of various human disorders. To develop more drug-like cHAUs for in vivo studies and drug development, the present application discloses bioassay guided structure—activity relationship studies, replacing the cyclohexyl ring with various 4-6-membered rings and further substitutions on the N-phenyl ring. The compounds described herein were tested in surrogate eIF2α phosphorylation and cell proliferation assays, and a subset of compounds were further tested in secondary mechanistic assays that included endogenous eIF2α phosphorylation and expression of C/EBP homologous protein (CHOP), a downstream effector. Specificity of the compounds for HRI was analyzed by testing the anti-proliferative activity in cells transfected with siRNA targeting HRI or mock. As described herein, the tested compounds of the present application had significantly improved cLogPs with no loss of potencies.

Eukaryotic translation initiation factor 2 alpha (eIF2α) kinases play roles in responding to various stress conditions and adapting cellular metabolism to extracellular cues. Heme regulated eIF2α kinase, also known as heme regulated inhibitor (HRI), was first of four eIF2α kinases to be discovered. HRI expression is highest in the red blood cell (RBC) precursors where it contributes to differentiation and maturation of myelogenic lineage into RBCs. In the RBC precursors, HRI is maintained in the inactive state by heme (see e.g., Igarashi et al, J. Biol. Chem. 283 (2008) 18782-18791). Low levels of free heme lead to HRI activation through autophosphorylation (see e.g., Igarashi et al, FEBS J. 278 (2011) 918-928) which results in phosphorylating eIF2α. Phosphorylated eIF2α reduces the level of the translation initiation complex formed by eIF2, GTP and Met-tRNAᵢ (the ternary complex, eIF2·GTP·Met-tRNAᵢ), which is critical for the formation of the 43S pre-initiation complex (see e.g., de la Parra et al, Curr. Opin. Genet. Dev. 48 (2018) 82-88). Reducing the amount of the ternary complex inhibits translation initiation thereby reducing global protein synthesis. By coupling globin synthesis to heme availability, HRI plays a critical role in attenuating severity of iron-deficiency anemia, β-thalassemia, and other anemic disorders (see e.g., Han et al, J. Clin. Invest. 115 (2005) 1562-1570.

HRI expression is not limited to myelogenic lineage; it is expressed in almost all tissues examined. HRI is the only eIF2α kinase activated by arsenate induced oxidative stress (see e.g., Suragani et al, Blood, 119 (2012) 5276-5284. It is also activated by nitrous oxide (see e.g., Igarashi et al, J. Biol. Chem. 279 (2004) 15752-15762, osmotic shock, and heat shock (see e.g., Berwal et al, Int. J. Biol. Macromol. 118 (2018) 1604-1613). By phosphorylating eIF2α and reducing the amount of ternary complex, HRI activates downstream effectors of this pathway including activating transcription factor 4 (ATF-4) and pro-apoptotic transcription factor C/EBP homology protein (CHOP). Activation of HRI (and the resulting phosphorylation of eIF2α) may initially be cytoprotective but its sustained activation may cause cell death (see e.g., Burwick et al, Expert Opin. Ther. Targets, 21 (2017) 1171-1177).

Recent discovery of small molecule activators of HRI (see e.g., Chen et al, Nat. Chem. Biol. 7 (2011) 610-616; Denoyelle et al, Bioorg. Med. Chem. Lett. 22 (2012) 402-409; Chen et al, J. Med. Chem. 56 (2013) 9457-9470; and Yefidoff-Freedman et al, J. Med. Chem. 60 (2017) 5392-5406) provided tools to better understand the role of this kinase as well as eIF2α phosphorylation in normal- and patho-biology. For example, these agents for the study HRI's regulation of fibroblast growth factor 21 (FGF21) activity and its role in diabetes and non-alcoholic fatty liver disease, and interaction of this HRI/eIF2α-P/ATF-4 pathway with the PPAR-β/δ pathway (see e.g., Zarei et al, Diabetes, (2016) db160155; and Zarei et al, Mol. Metab. 8 (2018) 117-131), regulation of host/intracellular pathogen interactions (see e.g., Infect. Immun. 86 (2018) e00707-17; and Machado et al, Sci. Rep. 8 (2018) 4857), and treatment of therapy resistant multiple myeloma cancers (see e.g., Burwick et al, Expert Opin. Ther. Targets, 21 (2017) 1171-1177; and Burwick et al, Leuk. Res. 55 (2017) 23-32). When employed in combination with other eIF2α kinase activators, HRI activators are useful tools for dissecting contribution of eIF2α compared to other substrates of eIF2α kinases to normal- and patho-biology. This cannot be accomplished by using non-specific eIF2α activators or eIF2α phosphatase inhibitors (see e.g., Aktas et al, Oncotarget, 4 (2013) 1606-1617; and Aktas et al, Oncotarget, 6 (2015) 6902-6914). Understanding the molecular basis of HRI activation by small molecules will be useful for understanding diverse roles heme and heme regulated proteins in normal- and patho-physiology (see e.g., Shimizu et al, Chem. Soc. Rev. 48 (2019) 5624-5657).

Modification of eIF2α phosphorylation is a viable approach for the treatment of animal models of proliferative and some neurodegenerative disorders (see e.g., Aktas et al, Oncotarget, 4 (2013) 1606-1617; Aktas et al, Oncotarget, 6 (2015) 6902-6914; Sidrauski et al, eLife, 4 (2015) e05033; Tenkerian et al, Mol. Cancer Res. 13 (2015) 1377-1388; Wang et al, Hum. Mol. Genet. 23 (2014) 2629-2638; and Kim et al, Nat. Genet. 46 (2013) 152-160). Previous reports describe screening of a N,N′-disubstituted urea library, 1-phenyl-3-(4-phenoxy)cyclohexyl)ureas (ΦOcHΦUs) as potent in vitro inducers of HRI-dependent phosphorylation, and a limited structure-activity relationship (SAR) study (see e.g., Chen et al, J. Med. Chem. 56 (2013) 9457-9470). Design and synthesis of ΦOcHΦUs led to compounds with improved biophysical properties that potently induced eIF2α phosphorylation and expression of its downstream effector CHOP and inhibit cancer cell proliferation at sub-micromolar concentrations (see e.g., Yefidoff-Freedman et al, J. Med. Chem. 60 (2017) 5392-5406). One of the ΦOcHΦUs with more potent in vitro activity, (1-(3-cyano-5-trifluoromethyl) phenyl-3-(trans-4-(4-(trifluoromethyl)phenoxy)cyclohexyl)urea, 1-VI in Table 1), displayed in vivo efficacy in xenograft mice model of human melanoma with no apparent organ toxicity and a very encouraging target validation profile. Recent reports have also described the ΦOcHΦU chemotype in which a combination of N-(3-trifluoromethyl-5-cyano)phenyl and N-(1,4-trans)-(4-(4-trifluoromethyl) phenoxy)cyclohexyl) moieties contributed to enhanced potency and favorable cLogP values (see e.g., Yefidoff-Freedman et al, J. Med. Chem. 60 (2017) 5392-5406).

The present application describes new compounds that activate eIF2α phosphorylation and its downstream effectors, and potently inhibit cancer cells proliferation.

Abbreviations and Definitions

ΦOcHΦUs: 1-phenyl-3-(4-phenoxy)cyclohexyl)ureas;

ΦOcAlkΦUs: 1-phenyl-3-(4-phenoxy)cycloalkyl)ureas;

4-CF₃-ΦOcAlkΦUs: 1-phenyl-3-(1,3/4-trans)-4-(4-trifluoromethyl) phenoxy)cycloalky)ureas;

4-CF₃-ΦOcBΦUs: 1-phenyl-3-((1,3-trans)-4-(4-trifluoromethyl) phenoxy)cyclobutyl)ureas;

4-CF₃-ΦcPΦUs: 1-phenyl-3-((1,3-trans)-4-(4-trifluoromethyl) phenoxy)cyclopentyl)ureas;

4-CF₃-ΦcHΦUs: 1-phenyl-3-((1,4-trans)-4-(4-trifluoromethyl) phenoxy)cyclohexyl)ureas;

4-CF₃-ΦPyΦUs: 1-phenyl-3-(5-(4-(trifluoromethyl)phenoxy)pyrimidin-2-yl)ureas;

HRI: heme regulated inhibitor or heme regulated eIF2α kinase;

eIF2: eukaryotic translation initiation factor 2;

eIF2α: eukaryotic translation initiation factor 2 alpha;

GTP: guanosine triphosphate;

Met-tRNAᵢ: initiator methionyl tRNA;

mRNA: messenger RNA;

ATF-4: activating transcription factor 4;

CHOP: C/EBP homology protein;

PPAR-β/δ: peroxisome proliferator-activated receptor (β/δ;

SAR: structure-activity relationship;

LC-MS: liquid chromatography-mass spectrometry;

NMR: nuclear magnetic resonance;

DIAD: diisopropyl azodicarboxylate;

TFA: trifluoroacetic acid;

BOC: tert-butyloxycarbonyl;

DMF: N,N-dimethylformamide;

NMP: N-methyl-2-pyrrolidinone;

DMSO: dimethyl sulfoxide;

DLR assay: surrogate dual-luciferase reporter eIF2α phosphorylation assay;

ORFs: upstream open reading frames;

5′UTR: 5′ untranslated region;

Cycloalk. config.: Cycloalkyl configuration.

Compounds

The present application provides compounds of Formula I:

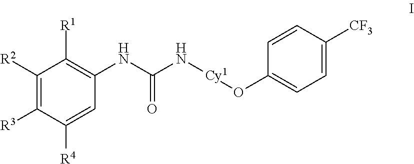

or pharmaceutically acceptable salts thereof, wherein:

Cy¹ is selected from the group consisting of a C₃₋₁₀ cycloalkyl ring and a 5-10 membered heteroaryl ring; and

R¹, R², R³, and R⁴ are each independently selected from the group consisting of H, halo, C₁₋₆ alkyl, C₁₋₆ haloalkyl, and cyano.

In some embodiments, at least one of R¹, R², R³, and R⁴ is not H.

In some embodiments, the compound of Formula I is not selected from the group consisting of:

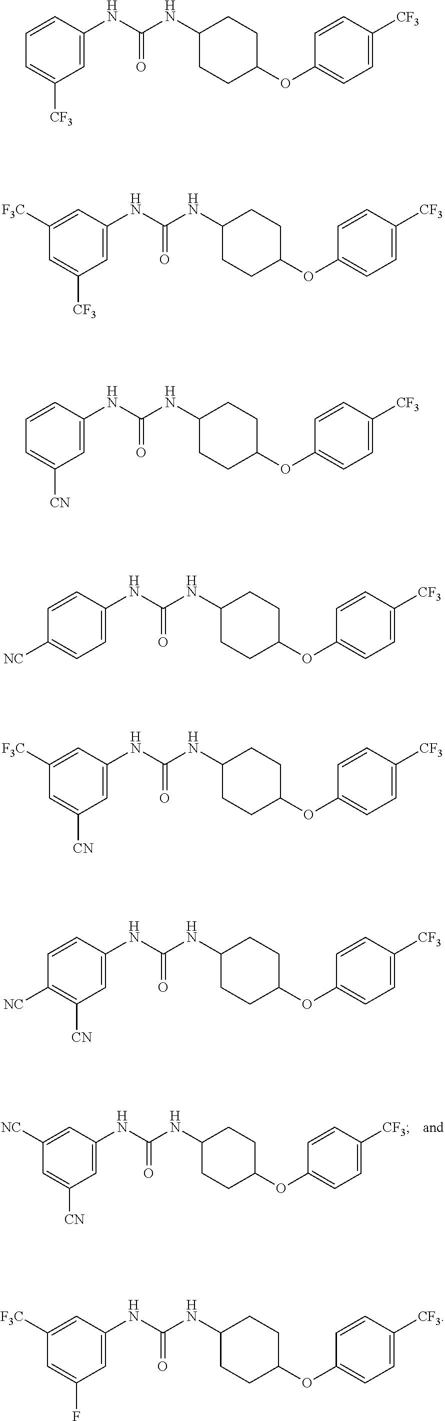

In some embodiments, the compound of Formula I is not a compound disclosed in U.S. Publication No.: US 20160318856, the disclosure of which is incorporated herein by reference in its entirety.

In some embodiments, Cy¹ is a C₃₋₁₀ cycloalkyl ring. In some embodiments, Cy¹ is a C₃₋₆ cycloalkyl ring. In some embodiments, Cy¹ is selected from the group consisting of cyclobutyl, cyclopentyl, and cyclohexyl. In some embodiments, Cy¹ is selected from the group consisting of cyclobut-1,3-diyl, cyclopent-1,3-diyl, and cyclohex-1,4-diyl. In some embodiments, Cy¹ is cyclobut-1,3-diyl. In some embodiments, Cy¹ is cyclopent-1,3-diyl. In some embodiments, Cy¹ is cyclohex-1,4-diyl.

In some embodiments, Cy¹ is a 5-10 membered heteroaryl ring. In some embodiments, Cy¹ is a 5-6 membered heteroaryl ring. In some embodiments, Cy¹ is a 6-membered heteroaryl ring. In some embodiments, Cy¹ is pyrimidinyl.

In some embodiments, R¹ is selected from the group consisting of H, halo, and C₁₋₆ alkyl. In some embodiments, R¹ is selected from the group consisting of H, halo, and C₁₋₃ alkyl. In some embodiments, R¹ is selected from the group consisting of H, halo, and C₁-₃ alkyl. In some embodiments, R¹ is selected from the group consisting of H, fluoro, and methyl. In some embodiments, R¹ is H. In some embodiments, R¹ is halo. In some embodiments, R¹ is fluoro. In some embodiments, R¹ is C₁₋₆ alkyl. In some embodiments, R¹ is C₁₋₃ alkyl. In some embodiments, R¹ is methyl.

In some embodiments, R² is selected from the group consisting of H, halo, C₁₋₃ alkyl, C₁₋₃ haloalkyl, and cyano. In some embodiments, R² is selected from the group consisting of H, fluoro, C₁₋₆ alkyl, C₁₋₆ haloalkyl, and cyano. In some embodiments, R² is selected from the group consisting of H, fluoro, C₁₋₃ alkyl, C₁₋₃ haloalkyl, and cyano. In some embodiments, R² is selected from the group consisting of H, fluoro, methyl, trifluoromethyl, and cyano. In some embodiments, R² is H. In some embodiments, R² is halo. In some embodiments, R² is fluoro. In some embodiments, R² is C₁₋₆ alkyl. In some embodiments, R² is C₁₋₃ alkyl. In some embodiments, R² is methyl. In some embodiments, R² is C₁₋₆ haloalkyl. In some embodiments, R² is C₁₋₆ fluoroalkyl. In some embodiments, R² is C₁₋₃ haloalkyl. In some embodiments, R² is C₁₋₃ fluoroalkyl. In some embodiments, R² is trifluoromethyl. In some embodiments, R² is cyano.

In some embodiments, R³ is selected from the group consisting of H, halo, and cyano. In some embodiments, R³ is selected from the group consisting of H, fluoro, and cyano. In some embodiments, R³ is H. In some embodiments, R³ is halo. In some embodiments, R³ is fluoro. In some embodiments, R³ is cyano.

In some embodiments, R⁴ is selected from the group consisting of H, halo, and cyano. In some embodiments, R⁴ is selected from the group consisting of H, fluoro, and cyano. In some embodiments, R⁴ is H. In some embodiments, R⁴ is halo. In some embodiments, R⁴ is fluoro. In some embodiments, R⁴ is cyano.

In some embodiments:

Cy¹ is selected from the group consisting of a C₃₋₁₀ cycloalkyl ring and a 5-10 membered heteroaryl ring;

R¹ is selected from the group consisting of H, halo, and C₁₋₆ alkyl;

R² is selected from the group consisting of H, halo, C₁₋₆ alkyl, C₁₋₆ haloalkyl, and cyano;

R³ is selected from the group consisting of H, halo, and cyano; and

R⁴ is selected from the group consisting of H, halo, and cyano.

In some embodiments:

Cy¹ is selected from the group consisting of a C₃₋₆ cycloalkyl ring and a 5-6 membered heteroaryl ring;

R¹ is selected from the group consisting of H, halo, and C₁₋₆ alkyl;

R² is selected from the group consisting of H, halo, C₁₋₆ alkyl, C₁₋₆ haloalkyl, and cyano;

R³ is selected from the group consisting of H, halo, and cyano; and

R⁴ is selected from the group consisting of H, halo, and cyano.

In some embodiments:

Cy¹ is selected from the group consisting of a C₃₋₁₀ cycloalkyl ring and a 5-10 membered heteroaryl ring;

R¹ is selected from the group consisting of H, halo, and C₁₋₃ alkyl;

R² is selected from the group consisting of H, halo, C₁₋₃ alkyl, C₁₋₃ haloalkyl, and cyano;

R³ is selected from the group consisting of H, halo, and cyano; and

R⁴ is selected from the group consisting of H, halo, and cyano.

In some embodiments:

Cy¹ is selected from the group consisting of a C₃₋₆ cycloalkyl ring and a 5-6 membered heteroaryl ring;

R¹ is selected from the group consisting of H, halo, and C₁₋₃ alkyl;

R² is selected from the group consisting of H, halo, C₁₋₃ alkyl, C₁₋₃ haloalkyl, and cyano;

R³ is selected from the group consisting of H, halo, and cyano; and

R⁴ is selected from the group consisting of H, halo, and cyano.

In some embodiments:

Cy¹ is selected from the group consisting of a C₃₋₁₀ cycloalkyl ring and a 5-10 membered heteroaryl ring;

R¹ is selected from the group consisting of H, fluoro, and C₁₋₃ alkyl;

R² is selected from the group consisting of H, fluoro, C₁₋₃ alkyl, C₁₋₃ haloalkyl, and cyano;

R³ is selected from the group consisting of H, fluoro, and cyano; and

R⁴ is selected from the group consisting of H, fluoro, and cyano.

In some embodiments:

Cy¹ is selected from the group consisting of a C₃₋₆ cycloalkyl ring and a 5-6 membered heteroaryl ring;

R¹ is selected from the group consisting of H, fluoro, and C₁₋₃ alkyl;

R² is selected from the group consisting of H, fluoro, C₁₋₃ alkyl, C₁₋₃ haloalkyl, and cyano;

R³ is selected from the group consisting of H, fluoro, and cyano; and

R⁴ is selected from the group consisting of H, fluoro, and cyano.

In some embodiments:

Cy¹ is selected from the group consisting of a cyclobutyl, cyclopentyl, cyclohexyl, and pyrimidinyl;

R¹ is selected from the group consisting of H, fluoro, and C₁₋₃ alkyl;

R² is selected from the group consisting of H, fluoro, C₁₋₃ alkyl, C₁₋₃ haloalkyl, and cyano;

R³ is selected from the group consisting of H, fluoro, and cyano; and

R⁴ is selected from the group consisting of H, fluoro, and cyano.

In some embodiments:

Cy¹ is selected from the group consisting of a cyclobut-1,3-diyl, cyclopent-1,3-diyl, and cyclohex-1,4-diyl, and pyrimidinyl;

R¹ is selected from the group consisting of H, fluoro, and C₁₋₃ alkyl;

R² is selected from the group consisting of H, fluoro, C₁₋₃ alkyl, C₁₋₃ haloalkyl, and cyano;

R³ is selected from the group consisting of H, fluoro, and cyano; and

R⁴ is selected from the group consisting of H, fluoro, and cyano.

In some embodiments, two or three of R¹, R², R³, and R⁴ are H. In some embodiments, two of R¹, R², R³, and R⁴ are H. In some embodiments, three of R¹, R², R³, and R⁴ are H.

In some embodiments, the compound of Formula I is a compound of Formula II:

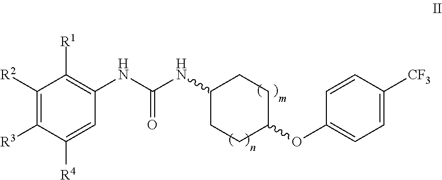

wherein n and m are each independently 0, 1, 2, or 3.

In some embodiments, the compound of Formula I is a compound of Formula IIa:

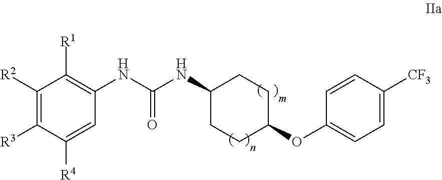

wherein n and m are each independently 0, 1, 2, or 3.

In some embodiments, the compound of Formula I is a compound of Formula IIb:

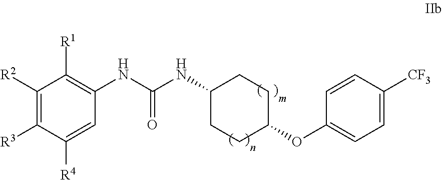

wherein n and m are each independently 0, 1, 2, or 3.

In some embodiments, the compound of Formula I is a compound of Formula IIc:

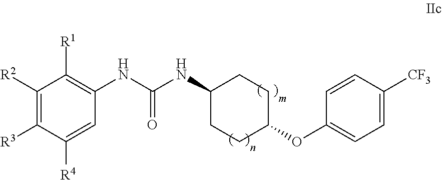

wherein n and m are each independently 0, 1, 2, or 3.

In some embodiments, the compound of Formula I is a compound of Formula IId:

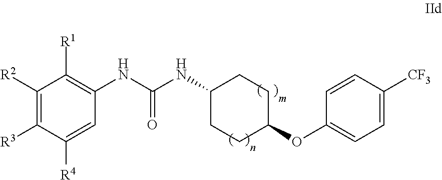

wherein n and m are each independently 0, 1, 2, or 3.

In some embodiments, the compound of Formula I is a compound of Formula III:

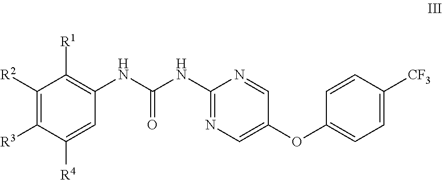

or a pharmaceutically acceptable salt thereof.

In some embodiments, the compound of Formula I is a compound of Formula IVa:

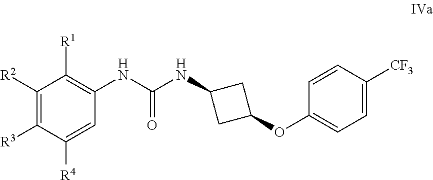

or a pharmaceutically acceptable salt thereof.

In some embodiments, the compound of Formula I is a compound of Formula IVb:

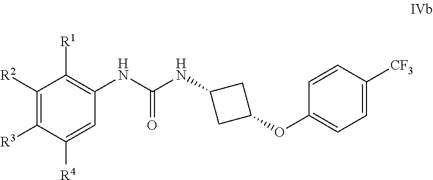

or a pharmaceutically acceptable salt thereof.

In some embodiments, the compound of Formula I is a compound of Formula IVc:

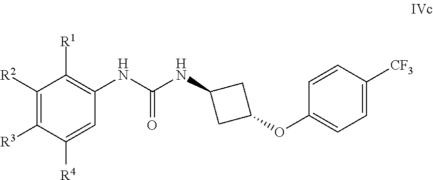

or a pharmaceutically acceptable salt thereof.

In some embodiments, the compound of Formula I is a compound of Formula IVd:

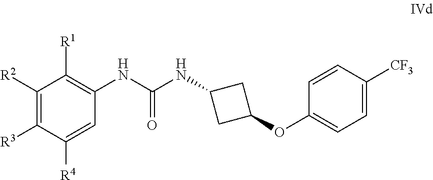

or a pharmaceutically acceptable salt thereof.

In some embodiments, the compound of Formula I is a compound of Formula Va:

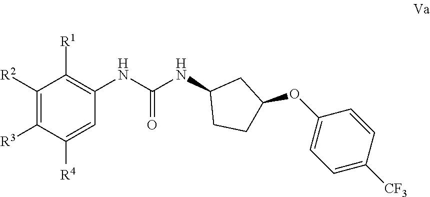

or a pharmaceutically acceptable salt thereof.

In some embodiments, the compound of Formula I is a compound of Formula Vb:

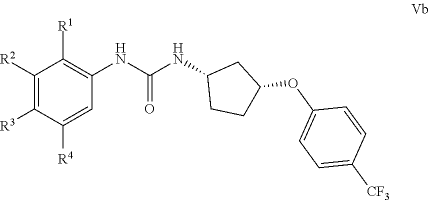

or a pharmaceutically acceptable salt thereof.

In some embodiments, the compound of Formula I is a compound of Formula Vc:

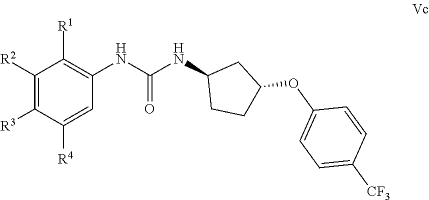

or a pharmaceutically acceptable salt thereof.

In some embodiments, the compound of Formula I is a compound of Formula Vd:

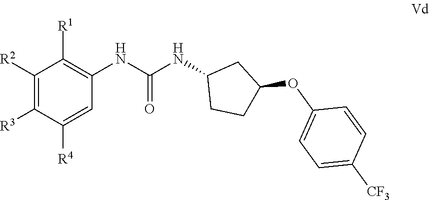

or a pharmaceutically acceptable salt thereof.

In some embodiments, the compound provided herein (e.g., the compound of Formula I), is selected from the group consisting of:

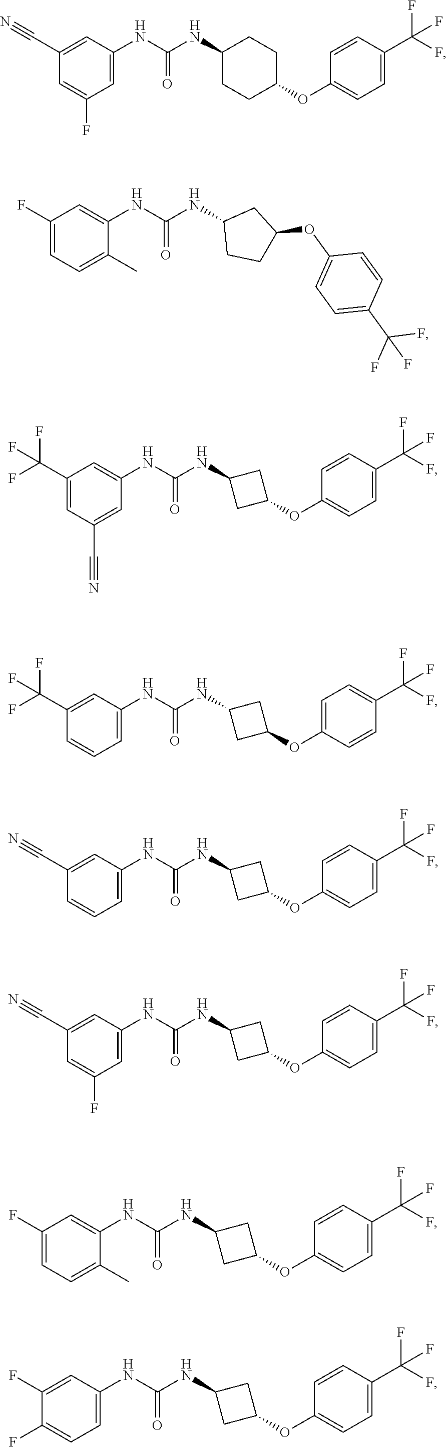

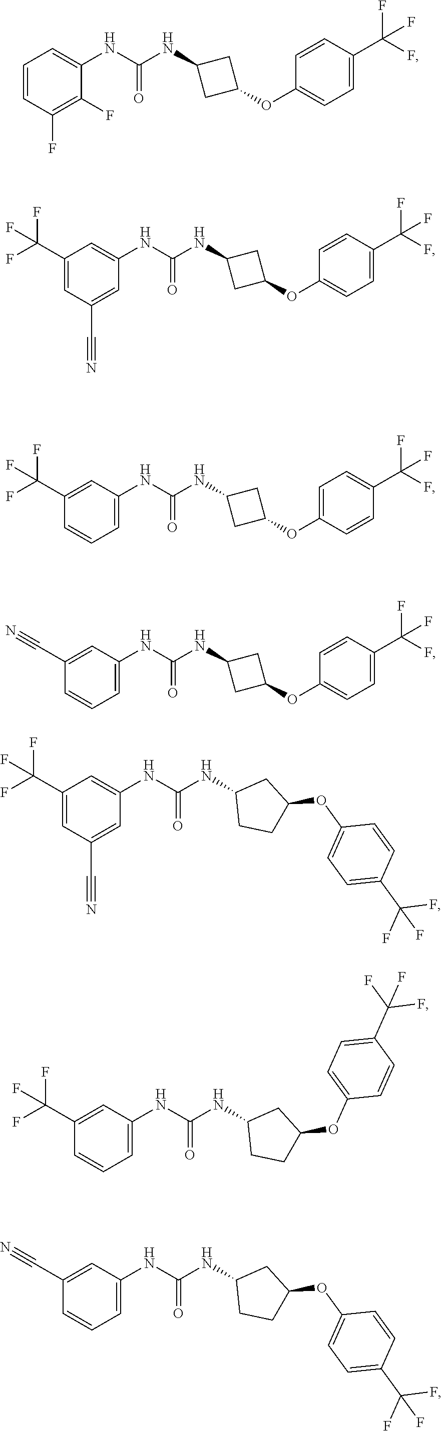

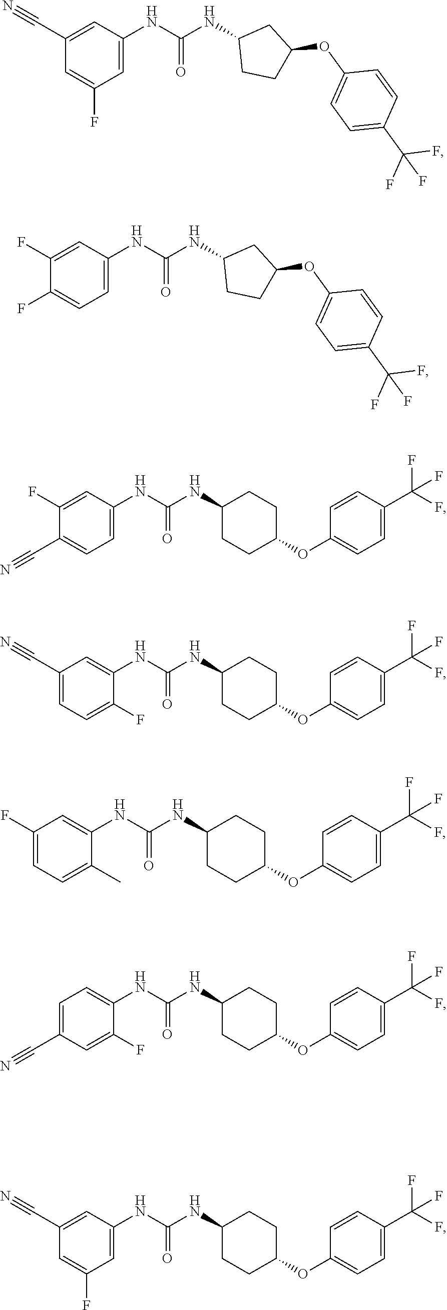

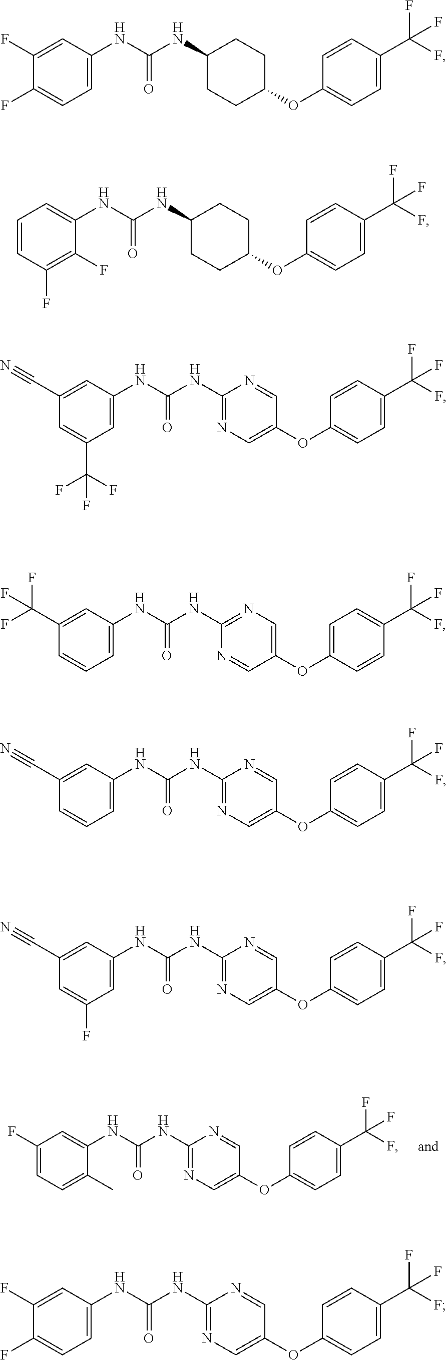

or a pharmaceutically acceptable salt thereof.

The term “n-membered” where n is an integer typically describes the number of ring-forming atoms in a moiety where the number of ring-forming atoms is n. For example, cyclohexyl is an example of a 6-membered cycloalkyl ring and pyrimidinyl is an example of a 6-membered heteroaryl ring.

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘalkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “halo” refers to fluoro, chloro, bromo, or iodo. In some embodiments, a halo is fluoro.

As used herein, the term “Cₙ₋ₘhaloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. Example haloalkyl groups include CF₃, C₂F₅, CHF₂, CH₂F, and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Cycloalkyl groups can have 3, 4, 5, 6, 7, 8, 9, or 10 ring-forming carbons (i.e., C₃₋₁₀). In some embodiments, the cycloalkyl is a C₃₋₁₀ cycloalkyl. In some embodiments, the cycloalkyl is a C₃₋₆ cycloalkyl. Example cycloalkyl groups include cyclopropyl, cyclobutyl, cyclopentyl, cyclohexyl, and the like.

As used herein, “heteroaryl” refers to a monocyclic or polycyclic (e.g., having 2 fused rings) aromatic heterocycle having at least one heteroatom ring member selected from N, O, and S. In some embodiments, the heteroaryl ring has 1, 2, 3, or 4 heteroatom ring members independently selected from N, O, and S. In some embodiments, the heteroaryl is a 5-10 membered heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl is a 5-6 monocyclic heteroaryl having 1 or 2 heteroatom ring members which are N. Example heteroaryl groups include, but are not limited to, thienyl (or thiophenyl), furyl (or furanyl), pyrrolyl, pyridinyl, pyrimidinyl, and the like.

At certain places, the definitions or embodiments refer to specific rings (e.g., a cyclohexyl ring). Unless otherwise indicated, these rings can be attached to any ring member provided that the valency of the atom is not exceeded. For example, an cycloalkyl ring may be attached at any position of the ring, whereas a cyclohex-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., compounds of Formula I, Formula II, Formula III, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H- 1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Synthesis

As will be appreciated by those skilled in the art, the compounds provided herein, including salts and stereoisomers thereof, can be prepared using known organic synthesis techniques and can be synthesized according to any of numerous possible synthetic routes. For example, the compounds provided herein, or intermediates useful in the preparation of the compounds provided herein, can be prepared according to the procedures described in one or more of Schemes 1-2, using appropriately substituted starting materials.

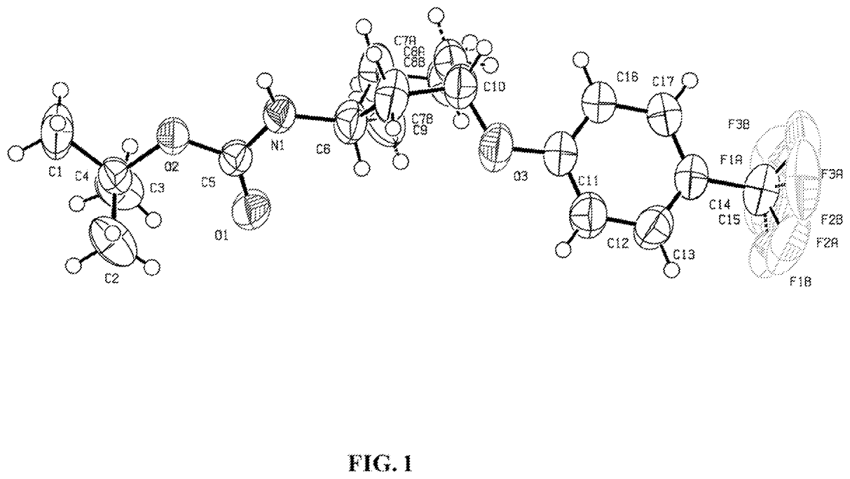

The common and principal intermediates for the synthesis of 1-phenyl-3-(4-phenoxy) cycloalkyl)ureas (ΦOcAlkΦUs) are the 3/4-(4-(trifluoromethyl) phenoxy)cycloalkan-1-amines (B; see Scheme 1). Synthesis of the B intermediates was accomplished by two different approaches:
(1). Mitsunobu coupling of a variety of tert-butyl (3/4-hydroxycycloalkyl) carbamates with 4-(trifluoromethyl)phenol in the presence of triphenylphosphine and diisopropyl azodicarboxylate (DIAD) in anhydrous THF afforded the Mitsunobu products, tert-butyl (3/4-(4-(trifluoromethyl) phenoxy)cycloalkyl)carbamates (A). Treatment of A with trifluoroacetic acid (TFA) removed the protecting group BOC (tert-butyloxycarbonyl) and afforded the amines (B) in good yields (Pathway AI; see e.g., International Application Publication No.: WO 2012072512). The Mitsunobu reaction provided a reliable method to inverting the configuration of secondary alcohol chiral carbon (see e.g., Mitsunobu et al, Synthesis, (1981) 1-28). Compound A-V was prepared from optically pure tert-butyl (1S,3R)-3-hydroxycyclopentyl carbamate (C, m=0, n=1). Single crystals of A-V were isolated from methylene chloride, and the absolute configuration was determined to be (1S,3S) by single crystal X-ray diffraction (Cambridge Crystallographic Data Centre 1871144; see FIG. 1). This result confirmed the inversion of configuration of the chiral center undergoing the Mitsunobu reaction.
(2). Direct O-alkylation of (1,4-trans)-4-aminocyclohexan-1-ol by 1-fluoro-4-(trifluoromethyl) benzene in the presence of NaH in DMF furnished the intermediate B (m=1, n=1) (Pathway AII; see e.g., Chen et al, J. Med. Chem. 56 (2013) 9457-9470; and Yefidoff-Freedman et al, J. Med. Chem. 60 (2017) 5392-5406).

With the common and principal intermediate B in hand, the N,N′-disubstituted ureas were prepared by acylation with either commercially available isocyanates D (see Pathway A, Scheme 1) or substituted phenyl carbamates E that were prepared from the corresponding anilines F and phenyl chloroformate (see Pathway B, Scheme 1).

Scheme 1

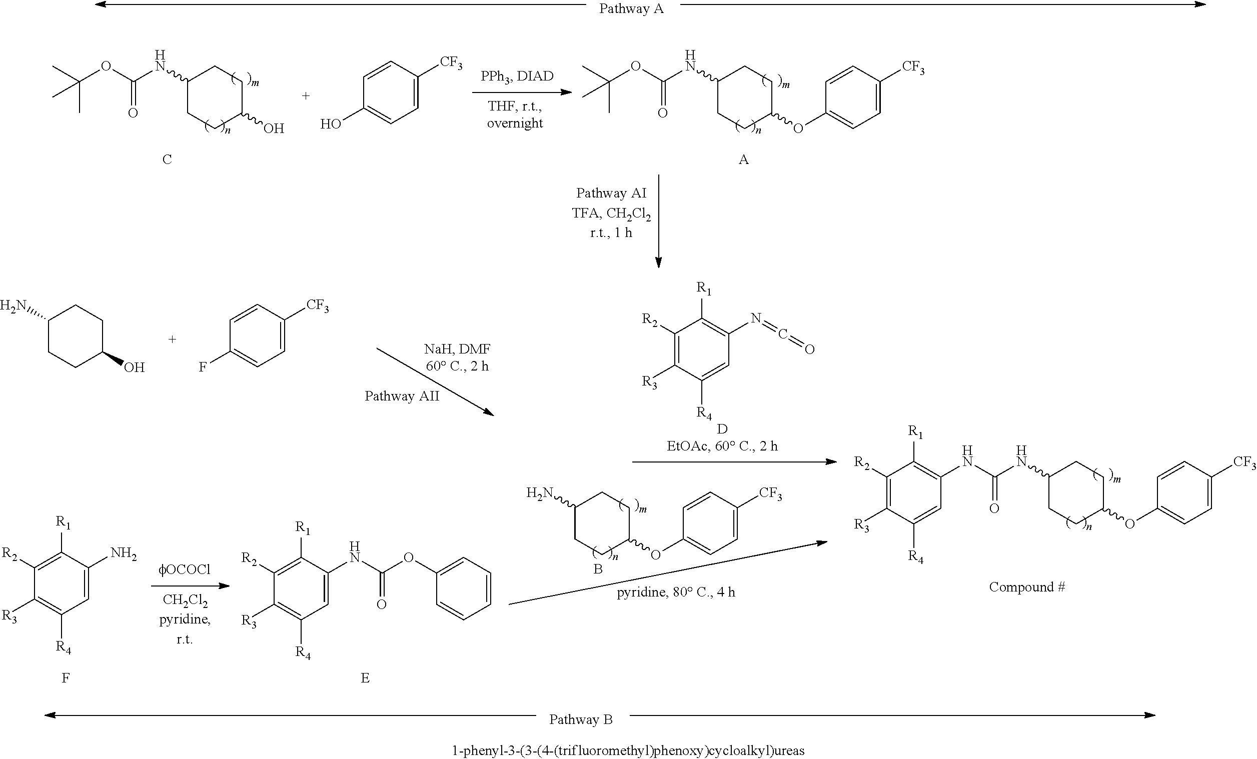

| Comp. | | Cycloalk. | | | | | | |
|---|---|---|---|---|---|---|---|---|
| # | Pathway | config. | m | n | R₁ | R₂ | R₃ | R₄ |
| 1-VI | B | 1,4-trans | 1 | 1 | H | CF₃ | H | CN |
| [12] | | | | | | | | |
| 1-V | B | 1S,3S | 0 | 1 | H | CF₃ | H | CN |
| 1-IV | B | 1,3-trans | 0 | 0 | H | CF₃ | H | CN |
| 1-IVc | B | 1,3-cis | 0 | 0 | H | CF₃ | H | CN |
| 2-VI | AII | 1,4-trans | 1 | 1 | H | CF₃ | H | H |
| [11] | | | | | | | | |
| 2-V | AI | 1S,3S | 0 | 1 | H | CF₃ | H | H |
| 2-IV | AI | 1,3-trans | 0 | 0 | H | CF₃ | H | H |
| 2-IVc | AI | 1,3-cis | 0 | 0 | H | CF₃ | H | H |
| 3-VI | AII | 1,4-trans | 1 | 1 | H | CN | H | H |
| [12] | | | | | | | | |
| 3-V | AI | 1S,3S | 0 | 1 | H | CN | H | H |
| 3-IV | AI | 1,3-trans | 0 | 0 | H | CN | H | H |
| 3-IVc | AI | 1,3-cis | 0 | 0 | H | CN | H | H |
| 4-VI | AII | 1,4-trans | 1 | 1 | H | F | H | CN |
| 4-V | AI | 1S,3S | 0 | 1 | H | F | H | CN |
| 4-IV | AI | 1,4-trans | 0 | 0 | H | F | H | CN |
| 5-VI | B | 1,4-trans | 1 | 1 | H | F | CN | H |
| 6-VI | B | 1,4-trans | 1 | 1 | F | H | H | CN |
| 7-VI | AII | 1,4-trans | 1 | 1 | CH₃ | H | H | F |
| 7-V | AI | 1S,3S | 0 | 1 | CH₃ | H | H | F |
| 7-IV | AI | 1,3-trans | 0 | 0 | CH₃ | H | H | F |
| 8-VI | B | 1,4-trans | 1 | 1 | F | H | CN | H |
| 9-VI | B | 1,4-trans | 1 | 1 | H | CN | F | H |
| 10-VI | AII | 1,4-trans | 1 | 1 | H | F | F | H |
| 10-V | AI | 1S,3S | 0 | 1 | H | F | F | H |
| 10-IV | AI | 1,3-trans | 0 | 0 | H | F | F | H |
| 11-VI | AII | 1,4-trans | 1 | 1 | F | F | H | H |
| 11-IV | AI | 1,3-trans | 0 | 0 | F | F | H | H |

The synthesis of 1-phenyl-3-(5-(4-(trifluoromethyl)phenoxy)pyrimidin-2-yl)ureas (4-CF₃-ΦOPyΦUs: 1-py-4-py, 7-py and 10-py) utilized Pathways AII and B as described in Scheme 2. The initial condensation of 5-chloropyrimidin-2-amine and 4-(trifluoromethyl)phenol in the presence of potassium hydroxide and potassium carbonate at elevated temperature in DMF or NMP failed to afford the intermediate 5-(4-(trifluoromethyl) phenoxy)pyrimidin-2-amine (G). Alternatively, 2-aminopyrimidin-5-ol was condensed with 1-fluoro-4-(trifluoromethyl)benzene. Due to the greater acidity of 2-aminopyrimidin-5-ol compared to (1,4-trans)-4-aminocyclohexan-1-ol, the NaH/DMF used for the formation of (1,4-trans)-4-(4-(trifluoromethyl)phenoxy) cyclohexan-1-amine (B, m=1, n=1) was replaced with Cₛ₂CO₃/DMSO for the formation of the intermediate G, as shown in Scheme 2.

Scheme 2

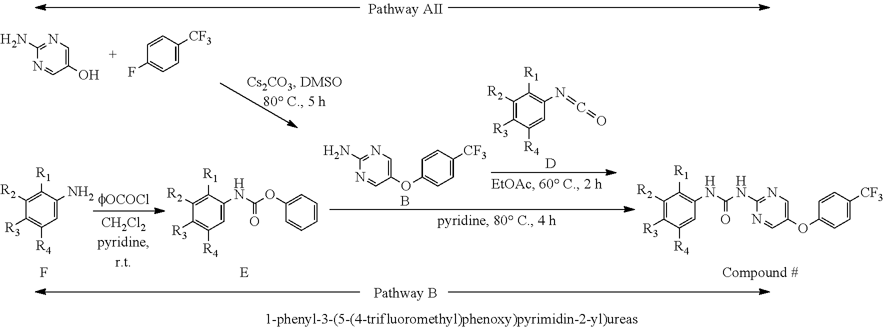

| Comp # | Pathway | R₁ | R₂ | R₃ | R₄ |
|---|---|---|---|---|---|
| 1-py | AII | H | CF₃ | H | CN |
| 2-py | AII | H | CF₃ | H | H |
| 3-py | B | H | CN | H | H |
| 4-py | B | H | F | H | CN |
| 7-py | AII | CH₂ | H | H | F |
| 10-py | AII | H | F | F | H |

A skilled artisan will appreciate that the processes described above are not the exclusive means by which compounds provided herein may be synthesized and that a broad repertoire of synthetic organic reactions is available to be potentially employed in synthesizing compounds provided herein. A skilled artisan knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Methods of Use

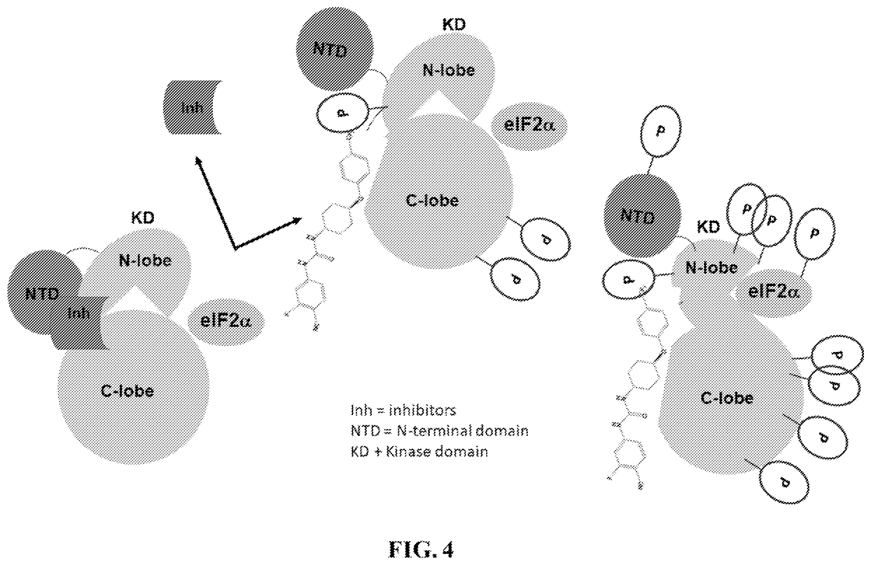

The methods described herein include methods for the treatment of disorders associated with an eIF2α kinase, eIF2α phosphorylation, uncontrolled translation initiation, or disorders that may be treated by inducing eIF2α phosphorylation. A hypothetical model of HRI activation by 4-CF₃-ΦOcHΦUs is shown in FIG. 4. The 4-CF₃-ΦOcHΦUs displace such inhibitors releasing the NTD from kinase domain which results in series of auto-phosphorylation events that change the relative orientation of N-lobe and C-lobe of kinase domain rendering the substrate binding domain accessible to eIF2α binding and catalysis.

In some embodiments, the present application provides methods of treating a disease in a patient (e.g., in a patient in need thereof), wherein the disease is associated with abnormal expression and/or activity of one or more eIF2α kinases. In some embodiments, the method comprises administering to the patient a therapeutically effective amount of a compound provided herein, or a pharmaceutically acceptable salt thereof. In some embodiments, the disease is associated with reduced expression and/or reduced activity of one or more eIF2α kinases. In some embodiments, the methods provided herein further comprise identifying a patient who has been diagnosed as having reduced expression and/or reduced activity of one or more eIF2α kinases.

Generally, the methods include administering a therapeutically effective amount of a compound as described herein, to a patient who is in need of, or who has been determined to be in need of, such treatment.

As used herein, the term “patient,” refers to any animal, including mammals such as mice, rats, other rodents, rabbits, dogs, cats, swine, cattle, sheep, horses, primates, and humans. In some embodiments, the patient is a human patient.

As used in this context, to “treat” means to ameliorate at least one symptom of the disorder associated with an eIF2α0 kinase, eIF2α phosphorylation, uncontrolled translation initiation, or disorders that may be treated by inducing eIF2α phosphorylation. In some embodiments, the disorder is selected from the group consisting of: a cancer, a hemolytic anemia, Wolcott-Rallison syndrome, a neurodegenerative disease, a motor neuron disease, tuberous sclerosis complex, an autism spectrum disorder, and a ribosomal defect disease.

In some embodiments, the disorder is a cancer. In some embodiments, the cancer is selected from the group consisting of: cervical cancer, liver cancer, bile duct cancer, eye cancer, esophageal cancer, head and neck cancer, brain cancer, prostate cancer, pancreatic cancer, skin cancer, testicular cancer, breast cancer, uterine cancer, penile cancer, small intestine cancer, colon cancer, stomach cancer, bladder cancer, anal cancer, lung cancer, lymphoma, leukemia, thyroid cancer, bone cancer, kidney cancer, and ovarian cancer. In some embodiments, the cancer is selected from the group consisting of: cervical cancer, liver cancer, glioblastoma, prostate cancer, pancreatic cancer, skin cancer, breast cancer, colon cancer, lung cancer, lymphoma, leukemia, kidney cancer, and ovarian cancer. In some embodiments, the cancer is selected from the group consisting of: breast cancer and skin cancer.

A method for selection of cancer patients for treatment is also provided. In accordance with certain examples, methods are provided of identifying cancer patients for treatment with compounds of Formulas (I). In some embodiments, cancer cells from a patient are assayed to determine the expression level of HRI. Based on the expression level of HRI, the patient is identified as a candidate for treatment with compounds Formula (I).

Once the HRI expression level of cancer cells from an individual is determined, such as by methods described herein, the individual may be identified as a suitable candidate for treatment with compounds Formula (I). According to one aspect, the compounds are administered to an individual in a manner to activate HRI thereby causing phosphorylation of eIF2α and inhibition of translation initiation.

In some embodiments, one or more compounds provided herein are used for the treatment of noncancereous cellular proliferative disorders. Examples of noncancerous cellular proliferative disorders includes fibroadenoma, adenoma, intraductal papilloma, nipple adenoma, adenosis, fibrocystic disease or changes of breast, plasma cell proliferative disorder (PCPD), restenosis, atherosclerosis, rheumatoid arthritis, myofibromatosis, fibrous hamartoma, granular lymphocyte proliferative disorders, benign hyperplasia of prostate, heavy chain diseases (HCDs), lymphoproliferative disorders, psoriasis, lung fibrosis (e.g., idiopathic pulmonary fibrosis), sclroderma, cirrhosis of the liver, IgA nephropathy, mesangial proliferative glomerulonephritis, membranoproliferative glomerulonephritis, hemangiomas, vascular and non-vascular intraocular proliferative disorders, polycythemia vera, pulmonary hypertension, and in-stent restenosis (see e.g., Grimminger F. et al., Nat. Rev. Drug Discov. (2010) 9(12):956-70).

The language “treatment of cellular proliferative disorders” is intended to include, but is not limited to, the prevention of the growth of neoplasms in a subject or a reduction in the growth of pre-existing neoplasms in a subject, as well as the prevention or reduction of increased or uncontrollable cell growth. The inhibition also can be the inhibition of the metastasis of a neoplasm from one site to another.

In some embodiments, the disorder is a hemolytic anemia, for example, a hemolytic anemia not caused by an infectious agent. In some embodiments, the hemolytic anemia is selected from erythropoietic protoporphyria, α-thalassemia, β-thalassemia, δ-thalassemia, sideroblastic anemia, and unstable hemoglobin hemolytic anemia. In some embodiments, the hemolytic anemia is β-thalassemia.

An assay for determining the effectiveness of a compound provided herein in treating a hemolytic anemia may be performed by contacting a cell with a compound provided herein, or a pharmaceutically acceptable salt form thereof, in vitro, and determining the effectiveness of the compound in inducing enhanced oxygen-carrying capacity in a cell in vitro. For example, human red blood progenitor cells may be obtained from human placenta cords discarded after birth or from β-thalassemia patients. CD34(+) cells may be separated by FACS (Fluorescent activated cell sorting), and induced to differentiate using erythropoietin. The cells may be treated with the compound or vehicle, and then evaluated at various stages of differentiation to red blood cells. The cell morphology, the ratio of mutant vs. wild-type hemoglobin, and the oxygen-carrying capacity of the differentiated red blood cells would be determined. A therapeutically effective amount would increase expression of wild-type hemoglobin and/or oxygen-carrying capacity of the cells treated with the compound compared to vehicle.

In some embodiments, the compounds may not change the ratio of mutant to wild type hemoglobin but may induce cells to fold the mutant protein similar to wild type configuration.

An assay for determining the effectiveness of a compound provided herein in treating a hemolytic anemia may be performed with an appropriate animal model and a compound provided herein, or a pharmaceutically acceptable salt form thereof, in vivo, and determining the effectiveness in inducing enhanced oxygen-carrying capacity in an animal in vivo. For example, several models of hemolytic anemia may be used, such as mutant β-thalassemia expressing cells, for in vivo studies. In such a mouse colony, mutant and wild-type pups would be obtained by breeding heterozygous mice. Mouse pups would be fed milk containing the compound or vehicle. The cell morphology, the ratio of mutant vs. wild-type hemoglobin, and the oxygen-carrying capacity of the animals' red blood cells would be determined. A therapeutically effective amount would increase expression of wild-type hemoglobin and/or oxygen-carrying capacity with the compound compared to vehicle.

In some embodiments, the disorder is Wolcott-Rallison syndrome.

An assay for determining the effectiveness of a compound provided herein in treating Wolcott-Rallison syndrome may be determined with an appropriate animal model and a compound provided herein, or a pharmaceutically acceptable salt form thereof, in vivo. Mice deficient in PERK, the human gene inactivated in patients suffering from Walcott-Rallison syndrome, or Akita mice, exhibiting a mutation in the insulin gene, may be used in the in vivo assay. PERK mice colonies would be provided with wild-type, heterozygous, and homozygous PERK knockout genotypes. Each genotype group would be split into two groups, and each group treated with milk or food containing either the compound or the vehicle. The weight and growth parameters of the mouse pups would be recorded weekly. Blood glucose and insulin levels would be determined at various times after feeding Glucose processing capacity would be determined via a glucose tolerance test. Populations would be sacrificed on days 20, 40, 60 and 80 after birth. The pancreas, liver, and bones would be examined for morphology and presence of pancreatic (3-cells. Homozygous PERK gene knockout mice will be smaller, fail to thrive, and die off quicker if fed vehicle containing milk or food compared to those fed milk or food containing the compound. The vehicle-treated pups will have greater impaired glucose tolerance, reduced insulin secretion, diminished numbers of pancreatic (3-cells, and display greater skeletal abnormalities compared with the compound-treated pups.

In some embodiments, the disorder is a neurodegenerative or motor neuron disease. In some embodiments, the neurodegenerative or motor neuron disease is selected from the group consisting of: amyotrophic lateral sclerosis, Alzheimer's disease, Amytrophic Lateral Sclerosis, Parkinson's disease, and Huntington's disease. In some embodiments, the neurodegenerative disease is Alzheimer's disease.

In some embodiments, the disease or disorder is selected from the group consisting of diabetes, non-alcoholic fatty liver disease, and tuberous sclerosis complex. In some embodiments, the disease or disorder is diabetes. In some embodiments, the disease or disorder is non-alcoholic fatty liver disease. In some embodiments, the disorder is tuberous sclerosis complex.

Synaptic transmission, long term memory formation and consolidation are highly dependent on regulated protein synthesis, including protein synthesis regulated by eIF2α kinases. Deregulation of protein synthesis may lead to abnormalities in long term memory formation, consolidation, and reconsolidation leading to autism spectrum disorders in a context dependent manner.

In some embodiments, the disorder is autism spectrum disorder. In some embodiments, the autism spectrum disorder is selected from the group consisting of: Asperger's syndrome, autistic disorder, Rett syndrome, childhood disintegrative disorder, and pervasive developmental disorder, not otherwise specified (PDD-NOS).

Unregulated protein synthesis has also been implicated in defective long term memory formation, consolidation, and reconsolidation. Inability to break protein synthesis underlies mental retardation disorders such as fragile-X syndrome.

In some embodiments, the disorder is a mental retardation disorder. In some embodiments, the mental retardation disorder is fragile-X syndrome.

In some embodiments, the disorder is a ribosomal defect disease. In some embodiments, the ribosomal defect disease is selected from the group consisting of: Shwachman-Bodian-Diamond syndrome, Diamond Blackfan anemia, and cartilage hair hypoplasia.

A method for activating an eIF2α kinase in a cell is also provided herein, the method comprising contacting the cell with an effective amount of a compound provided herein. In some embodiments, the binding and activation of an eIF2α kinase results in higher phosphorylation of an eIF2α to balance hemoglobin synthesis to the hemoglobin folding capacity of the cells which, in turn, leads to increased oxygen-carrying capacity in the cell. The method of activating an eIF2α kinase in a cell may be performed by contacting the cell with an effective amount of a compound provided herein, or a pharmaceutically acceptable salt form thereof, in vitro, thereby inducing activation of an eIF2α kinase in a cell in vitro. Uses of such an in vitro methods of activating an eIF2α kinase include, but are not limited to use in a screening assay (for example, wherein a compound provided herein is used as a positive control or standard compared to compounds of unknown activity or potency in activating an eIF2α kinase). In some embodiments thereof, activating of an eIF2α kinase is performed in a red blood cell progenitor.

The method of activating an eIF2α kinase in a cell may be performed, for example, by contacting a cell (e.g., a CD34+ progenitor cell) with a compound provided herein, in vivo, thereby activating an eIF2α kinase in a patient in vivo. The contacting is achieved by causing a compound as provided herein, or a pharmaceutically acceptable salt form thereof, to be present in the patient in an amount effective to achieve activation of an eIF2α kinase. This may be achieved, for example, by administering an effective amount of a compound provided herein, or a pharmaceutically acceptable salt form thereof, to a patient. Uses of such an in vivo methods of activating an eIF2α kinase include, but are not limited to, use in methods of treating a disease or condition, wherein activating an eIF2α kinase is beneficial. In some embodiments thereof, activation of an eIF2α kinase results in increased phosphorylation of an eIF2α kinase, and thereby greater oxygen-carrying capacity in a red blood cell, for example in a patient suffering from β-thalassemia or a related disorder. In some embodiments, the method is performed by administering a therapeutically effective amount of a compound provided herein, or a pharmaceutically acceptable salt form thereof, to a patient who is suffering from β-thalassemia or a related disorder.

As used herein, the term “contacting” refers to the bringing together of indicated moieties in an in vitro system or an in vivo system. For example, “contacting” a eIF2α kinase with a compound provided herein includes the administration of a compound provided herein, or a pharmaceutically acceptable salt thereof, to an individual or patient (e.g., a human patient), having an eIF2α kinase, as well as, for example, introducing a compound described herein into a sample containing a cellular or purified preparation containing the eIF2α kinase. In some embodiments, the compounds provided herein are selective activators of one or more eIF2α kinases.

As used herein, the phrase “therapeutically effective amount” refers to the amount of active compound or pharmaceutical composition (e.g., an amount of any solid form or salt thereof as provided herein) that elicits the biological or medicinal response in a tissue, system, animal, individual or human that is being sought by a researcher, veterinarian, medical doctor, or other clinician. An appropriate “effective” amount in any individual case may be determined using techniques known to a skilled artisan.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In some embodiments, each component is “pharmaceutically acceptable” as defined herein (see e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009).

In some embodiments, the compounds provided herein, and pharmaceutically acceptable salts thereof, may be useful in preventing or reducing the risk of developing any of the diseases referred to herein; e.g., preventing or reducing the risk of developing a disease, condition, or disorder in a patient who may be predisposed to the disease, condition, or disorder but does not yet experience or display the pathology or symptomatology of the disease.

Pharmaceutical Compositions and Formulations

When employed as pharmaceuticals, the compounds and salts provided herein can be administered in the form of pharmaceutical compositions. These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. In some embodiments, the compounds, salts, and pharmaceutical compositions provided herein are suitable for parenteral administration. In some embodiments, the compounds, salts, and pharmaceutical compositions provided herein are suitable for intravenous administration.

In some embodiments, the compounds, salts, and pharmaceutical compositions provided herein are suitable for oral administration.

Also provided are pharmaceutical compositions which contain, as the active ingredient, a compound provided herein, or a pharmaceutically acceptable salt thereof, in combination with one or more pharmaceutically acceptable carriers (e.g., excipients). In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active ingredient can be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

General Methods and Materials

All reagents and solvents were purchased from commercial sources and used as-is. Analytical HPLC was run on a Waters Alliance 2695 using a reverse-phase column (XBridge BEH130 C18, 4.6×100 mm, particle size 5 μm) eluting with a linear gradient of acetonitrile in water containing 0.1% trifluoroacetic acid (TFA). The purity of all target compounds was greater than 95% by analytical HPLC inspection. LC-MS analysis was run on a Waters Alliance 2695 with UV detector (214 and 254 nm) and Micromass ZQ quadrupole mass detector in electrospray positive (ESI⁺) mode using a reverse-phase column (Waters Symmetry C18, 2.1×100 mm, particle size 3.5 μm) eluting with a linear gradient of acetonitrile in water containing 0.1% formic acid. TLC analysis was run on Merck silica gel 60 F₂₅₄ aluminum sheets. Flash chromatography purifications were performed on Biotage SP1 using silica gel prepacked normal phase columns (200-400 mesh) eluting with a linear gradient of ethyl acetate in n-heptane, and fractions were collected at 254 nm and monitored at 280 nm. Melting points were determined on a Mel-Temp electrothermal apparatus equipped with a Barnaand thermometer and were uncorrected. Proton, carbon, and fluorine NMR experiments were performed on a Varian Inova 400 MHz spectrometer using DMSO-d₆ as solvent. Chemical shifts (δ) are reported in ppm relative to TMS as the internal standard. On the basis of analytical reverse-phase high-performance liquid chromatography (RP-HPLC) analysis, the purity of all final N,N′-disubstituted ureas submitted to biological characterization and reported herein equaled or exceeded 95%. Their structural identity and integrity were confirmed by LC-MS as well as ¹H-, ³C-, and ¹⁹F-NMR, as described in the Examples provided herein.

Plasmids and Ternary Complex Assay

The dual luciferase expression vector and other plasmids used in the Examples provided herein have been previously reported (see e.g., Ziegeler et al, J. Biol. Chem. 285 (2010) 15408-15419). The dual luciferase surrogate eIF2α phosphorylation assay, has also been previously reported (see e.g., Chen et al, Nat. Chem. Biol. 7 (2011) 610-616). Briefly, a dual Renilla and Firefly luciferase mammalian reporter vector that transcribes both mRNAs from the same bi-directional enhancer/promoter complex was utilized for generation of surrogate eIF2α phosphorylation assay (see e.g., Ziegeler et al, J. Biol. Chem. 285 (2010) 15408-15419). Both mRNAs contained the same 90 nucleotide plasmid derived 5′UTR. In addition, 5′UTR of the Firefly luciferase open reading frame was fused in-frame to the 267 nucleotide ATF-4 5′UTR (see e.g., Chen et al, Nat. Chem. Biol. 7 (2011) 610-616).

Dual Luciferase Reporter (DLR) Assay

Cells expressing firefly and renilla luciferases were assayed with a dual glow luciferase assay kit, per manufacturer's instruction (Promega Inc., Madison, WI). The data calculations were carried out as the ratio of firefly to renilla luciferase signal (see e.g., Ziegeler et al, J. Biol. Chem. 285 (2010) 15408-15419). Dose-response curves were obtained, and triplicate data points were fitted to the logistical sigmoidal model using nonlinear least-squares regression performed in GraphPad Prism 6.

Cell Lines and SiRNA Transfection

Stable cell lines utilized in this study were generated according to previous reports (see e.g., Bai et al, ChemBioChem, 14 (2013) 1255-1262). Briefly, cells were seeded at the density of 10⁵ in 60-mm dish and transfected one day later using the Lipofectamine 2000 (Invitrogen). For selection of stable cell lines, transfected cells were transferred to 100-mm plates and selected with appropriate antibiotics (see e.g., Chen et al, Nat. Chem. Biol. 7 (2011) 610-616). siRNA knockdown was carried out in 96-well plates by reverse transfection as previously reported (see e.g., Chen et al, Nat. Chem. Biol. 7 (2011) 610-616).

Western Blotting

Cells cultured under recommended media conditions were plated and maintained in serum-containing media without antibiotics in 14-cm plates (Nunc) until reaching 70% confluence. Cells were then treated with compounds for 6 h, washed with cold PBS once, and lysed with M-PER Mammalian Protein Extraction Reagent (Pierce) for 30 min on ice. The cell lysates were centrifuged at 12,000 RPM for 15 min and the supernatants were transferred to fresh tubes and the concentrations were determined by BCA (Pierce). Equal amount of proteins were mixed with Laemmli Sample Buffer, heated at 100° C. for 5 min, separated by SDS-PAGE, and probed with anti-phosphoserine-51-eIF2α (Phos-eIF2α), anti-total eIF2α-specific antibodies (Total-eIF2α) (Biosource International, Hopkinton, MA), anti-CHOP, anti-cyclin D1 or anti-actin (Santa Cruz Biotechnology, CA) using procedures substantially similar to previous reports (see e.g., Aktas et al, Mol. Cell. Biol. 17 (1997) 3850-3857).

Cell Growth Inhibition Assay

Cells were seeded in 96-well plates and maintained for 5 days in the presence of 0.5 μM to 20 μM of individual compound, and cell proliferation was measured by the sulforhodamine B (SRB) assay according to previous reports (see e.g., Palakurthi et al, Cancer Res. 60 (2000) 2919-2925): briefly, at the end of a 5-day treatment, cells were fixed in 10% cold trichloroacetic acid. Cell number was estimated by measuring the remaining bound dye of sulforhodamine B after washing. The percentage of growth was calculated by using the equation: 100×[(T-T₀)/(C-T₀)], where T and C represent the absorbance in treated and control cultures at Day 5, and T₀ at time zero, respectively. If T was less than T₀, cell death had occurred and was calculated from 100×[(T-T₀)/T₀].

Intermediate 1. tert-Butyl (1S,3S)-3-(4-(trifluoromethyl) phenoxy)cyclopentylcarbamate (A-V)

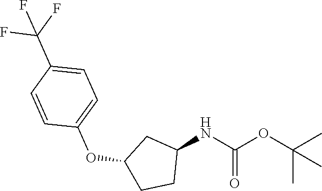 [SMILES: CC(C)(C)OC(=O)N[C@H]1CC[C@H](Oc2ccc(C(F)(F)F)cc2)C1]

To a solution of tert-butyl (1S,3R)-3-hydroxycyclopentylcarbamate (0.50 g, 2.5 mmol), 4-(trifluoromethyl)phenol (0.49 g, 3 mmol) and triphenylphosphine (0.98 g, 3.75 mmol) in anhydrous THF (8 mL) was added DIAD (0.98 mL, 4.68 mmol, dropwise) in anhydrous THF (7 mL). The reaction solution was stirred overnight at room temperature. The resulting solution was concentrated in vacuo, and the resulting residue was subjected to flash column chromatography on silica gel (eluting with EtOAc-heptane by a gradient of EtOAc from 4% to 32%) to afford the title compound as colorless crystals (0.63 g, 73%); mp 129.3-132.1° C. ¹H NMR (400 MHz, DMSO-d₆) δ7.60 (d, J=8.7 Hz, 2H), 7.04 (d, J=8.7 Hz, 2H), 6.93 (d, J=6.8 Hz, 1H), 4.96-4.86 (m, 1H), 3.95 (dq, J=13.8, 7.0 Hz, 1H), 2.15 (ddd, J=20.2, 8.8, 6.2 Hz, 1H), 1.94 (td, J=13.9, 7.3 Hz, 2H), 1.86-1.76 (m, 1H), 1.64 (ddd, J=10.2, 8.6, 2.4 Hz, 1H), 1.52-1.39 (m, 1H), 1.36 (s, 9H). ¹³C NMR (100 MHz, DMSO-d₆) δ160.78 (d, J=1.1 Hz), 155.51, 127.34 (q, J=3.7 Hz), 125.03 (q, J=270.8 Hz), 121.24 (q, J=32.1 Hz), 116.10, 78.16, 50.48, 30.85, 30.64, 28.70. ¹⁹F NMR (376 MHz, DMSO-d₆) δ−59.83.

Intermediate 2. tert-Butyl trans-3-(4-(trifluoromethyl)phenoxy)cyclobutylcarbamate (A-IV)

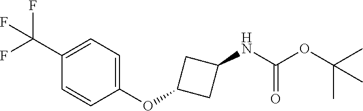 [SMILES: CC(C)(C)OC(=O)N[C@H]1C[C@H](Oc2ccc(C(F)(F)F)cc2)C1]

The title compound was prepared according to the procedures described in Intermediate 1. White solid (0.78 g, 94%); mp 137.8-141.8° C. ¹H NMR (400 MHz, DMSO-d₆) δ7.61 (d, J=6.8 Hz, 2H), 7.28 (d, J=5.0 Hz, 1H), 6.96 (d, J=6.9 Hz, 2H), 4.86 (d, J=2.7 Hz, 1H), 4.08 (d, J=4.6 Hz, 1H), 2.41-2.22 (m, 4H), 1.36 (d, J =2.4 Hz, 9H). ¹³C NMR (100 MHz, DMSO-d₆) δ160.50, 155.21, 127.42 (q, J=3.7 Hz), 124.99 (q, J=271.0 Hz), 121.57 (q, J=32.2 Hz), 115.71, 70.04, 42.05, 36.89, 28.66. ¹⁹F NMR (376 MHz, DMSO-d₆) δ−59.03.

Intermediate 3. tert-Butyl cis-3-(4-(trifluoromethyl) phenoxy)cyclobutylcarbamate (A-IVc)

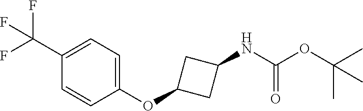 [SMILES: CC(C)(C)OC(=O)N[C@H]1C[C@@H](Oc2ccc(C(F)(F)F)cc2)C1]

The title compound was prepared according to the procedures described in Intermediate 1. White solid (0.45 g, 27%); mp 157.0-159.0° C. (CH₂C₁₂). ¹H NMR (400 MHz, DMSO-d₆) δ7.60 (d, J=7.6 Hz, 2H), 7.18 (d, J=7.2 Hz, 1H), 7.00 (d, J=7.8 Hz, 2H), 4.52-4.30 (m, 1H), 3.69 (dd, J=15.7, 8.0 Hz, 1H), 2.76 (d, J=6.3 Hz, 2H), 1.97 (d, J=8.2 Hz, 2H), 1.36 (s, 9H). ¹³C NMR (100 MHz, DMSO-d₆) δ160.38 (d, J=1.3 Hz), 155.02, 127.42 (q, J=3.7 Hz), 124.98 (q, J=270.6 Hz), 121.59 (q, J=32.1 Hz), 115.64, 65.68, 38.51, 37.65, 28.66. ¹⁹F NMR (376 MHz, DMSO-d₆) δ−59.11.

Intermediate 4. (1S,3S)-3-(4-(Trifluoromethyl)phenoxy)cyclopentanamine (B-V)

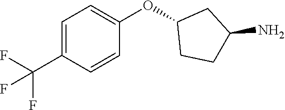 [SMILES: N[C@H]1CC[C@H](Oc2ccc(C(F)(F)F)cc2)C1]

Compound A-V (Intermediate 1, 518 mg, 1.5 mmol) was dissolved in anhydrous methylene chloride (10 mL), and a solution of trifluoroacetic acid (5.31 g, 45 mmol) in anhydrous methylene chloride (5 mL) was added. The reaction solution was stirred at room temperature for 1 h. The resulting solution was concentrated using a rotary evaporator, and the resulting residue was diluted in methylene chloride (60 mL), washed with saturated aqueous sodium bicarbonate (2×20 mL), dried over anhydrous sodium sulfate, and concentrated in vacuo to afford the title compound as semi-solid (380 mg, 103%), which was used in the next step without further purification. ¹H NMR (400 MHz, DMSO-d₆) δ7.59 (d, J=8.7 Hz, 2H), 7.02 (d, J=8.6 Hz, 2H), 4.93 (m, 2H), 3.41 (m, 1H), 2.22 (m, 1H), 2.01-1.81 (m, 2H), 1.76-1.66 (m, 1H), 1.66-1.55 (m, 1H), 1.29 (m, 1H), 1.39-1.17 (m, 1H). ¹⁹F NMR (376 MHz, DMSO-d₆) δ−59.77.

Intermediate 5. trans-3-(4-(Trifluoromethyl)phenoxy)cyclobutanamine (B-IV)

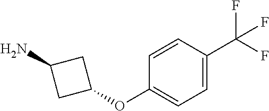 [SMILES: N[C@H]1C[C@H](Oc2ccc(C(F)(F)F)cc2)C1]

The title compound was prepared according to the procedures described in Intermediate 4. Yellow semi-solid (0.49 g, 100%). ¹H NMR (400 MHz, DMSO-d₆) δ7.60 (d, J=8.7 Hz, 2H), 6.95 (d, J=8.6 Hz, 2H), 4.99-4.77 (m, 2H), 3.70-3.45 (m, 1H), 2.25 (ddd, J=11.9, 7.5, 4.1 Hz, 2H), 2.14 (dt, J=12.3, 6.1 Hz, 2H). ¹³C NMR (100 MHz, DMSO-d₆) δ160.70, 127.39 (q, J=3.8 Hz), 125.02 (q, J=271.0 Hz), 121.37 (q, J=31.7 Hz), 115.70, 70.57, 43.93, 39.61. ¹⁹F NMR (376 MHz, DMSO-d₆) δ−59.84.

Intermediate 6. cis-3-(4-(Trifluoromethyl)phenoxy)cyclobutan-1-amine (B-IVc)

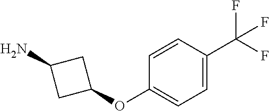 [SMILES: N[C@H]1C[C@@H](Oc2ccc(C(F)(F)F)cc2)C1]

The title compound was prepared according to the procedures described in Intermediate 4. Off white solid (0.28 g, 100%); mp 71.9-74.3° C.

Intermediate 7. 5-(4-(Trifluoromethyl)phenoxy)pyrimidin-2-amine (B-py)

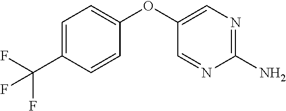 [SMILES: Nc1ncc(Oc2ccc(C(F)(F)F)cc2)cn1]

A mixture of 2-aminopyrimidin-5-ol (0.5 g, 4.5 mmol), 1-fluoro-4-(trifluoromethyl) benzene (0.74 g, 4.5 mmol) and cesium carbonate (4.4 g, 13.5 mmol) in DMSO (8 mL) was stirred under argon at 80° C. for 5 h. The resulting reaction mixture was diluted with ethyl acetate (90 mL) and water (30 mL). The aqueous phase was back-extracted with ethyl acetate (60 mL). The combined extract was washed with brine (5×30 mL), dried over anhydrous sodium sulfate overnight, and concentrated in vacuo. The resulting residue was recrystallized from hot ethyl acetate to afford the title compound as white crystalline solid (0.12 g, 10%); mp 132.5-134.5° C. (EtOAc). ¹H NMR (400 MHz, DMSO-d₆) δ8.20 (s, 2H), 7.68 (d, J=8.7 Hz, 2H), 7.10 (d, J=8.6 Hz, 2H), 6.73 (s, 2H). ¹³C NMR (100 MHz, DMSO-d₆) δ161.95 (d, J =1.3 Hz), 161.85, 151.94, 141.27, 127.85 (q, J=3.8 Hz), 124.73 (q, J=271.2 Hz), 123.38 (q, J=32.2 Hz), 116.78. ¹⁹F NMR (376 MHz, DMSO-d₆) δ−60.15.

Example 1. 1-(3-Cyano-5-fluorophenyl)-3-(trans-4-(4-(trifluoromethyl) phenoxy)cyclohexyl)urea (4-VI)

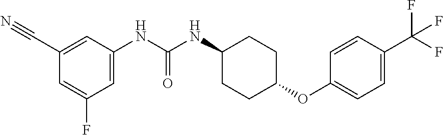 [SMILES: N#Cc1cc(F)cc(NC(=O)N[C@H]2CC[C@H](Oc3ccc(C(F)(F)F)cc3)CC2)c1]

Step 1. Phenyl (3-cyano-5-fluorophenyl)carbamate (E-4)

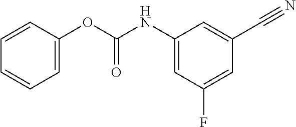 [SMILES: N#Cc1cc(F)cc(NC(=O)Oc2ccccc2)c1]

3-Amino-5-fluorobenzonitrile (0.41 g, 3 mmol) and anhydrous pyridine (0.36 g, 4.5 mmol) were dissolved in anhydrous methylene chloride (45 mL). Phenyl chloroformate (0.70 g, 4.5 mmol) was then added to the mixture, dropwise, at 0° C. The reaction mixture was stirred under argon at room temperature for 4 h. The resulting solution was washed with consecutively with 1 mol/L aqueous hydrochloride, water and brine, dried over anhydrous sodium sulfate, and concentrated in vacuo to afford phenyl (3-cyano-5-fluorophenyl)carbamate (E-4) as white solid in quantitative yield, which was used in the next step without further purification.

Step 2. 1-(3-Cyano-5-fluorophenyl)-3-(trans-4-(4-(trifluoromethyl) phenoxy)cyclohexyl)urea (4-VI)

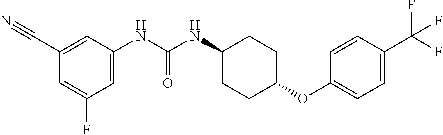 [SMILES: N#Cc1cc(F)cc(NC(=O)N[C@H]2CC[C@H](Oc3ccc(C(F)(F)F)cc3)CC2)c1]

A mixture of compound E-4 (0.13 g, 0.5 mmol) and trans-4-(4-(trifluoromethyl)phenoxy) cyclohexan-1-amine (B-VI) (0.13 g, 0.5 mmol) in anhydrous pyridine (9 mL) was stirred under argon at 80° C. for 4 h. The reaction solution was then concentrated in vacuo, and the resulting residue was crystallized from hot ethyl acetate to afford the title compound as crystalline white solid (105 mg, 50%); mp 237.8-240.8° C. (EtOAc). ¹H NMR (400 MHz, DMSO-d₆) δ8.84 (s, 1H), 7.64 (dt, J=11.8, 2.2 Hz, 1H), 7.59 (d, J=8.8 Hz, 2H), 7.58-7.57 (m, 1H), 7.28 (ddd, J=8.3, 2.3, 1.3 Hz, 1H), 7.11 (d, J=8.7 Hz, 2H), 6.47 (d, J=7.6 Hz, 1H), 4.52-4.36 (m, 1H), 3.59-3.47 (m, 1H), 2.16-1.95 (m, 2H), 1.95-1.79 (m, 2H), 1.58-1.27 (m, 4H). ¹³C NMR (100 MHz, DMSO-d₆) δ162.42 (d, J=243.3 Hz), 160.72 (d, J=1.1 Hz), 154.45, 143.82 (d, J=12.1 Hz), 127.36 (q, J=3.9 Hz), 125.01 (d, J=271.0 Hz), 121.27 (d, J=32.0 Hz), 118.30 (d, J=3.8 Hz), 117.29 (d, J=2.7 Hz), 116.26, 113.06 (d, J=12.3 Hz), 111.39 (d, J=25.6 Hz), 109.46 (d, J=26.3 Hz), 74.83, 47.74, 30.19, 29.94. ¹⁹F NMR (376 MHz, DMSO-d₆) δ−59.82, −109.68 (dd, J =11.7, 8.4 Hz). LC-MS (ESI): m/z 422.09[M+H]⁺, 463.10 [M+H+41]⁺.

Example 2. 1-(5-Fluoro-2-methylphenyl)-3-((1S,3S)-3-(4-(trifluoromethyl) phenoxy)cyclopentyl)urea (7-V)

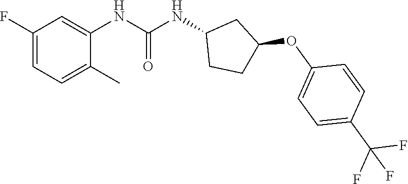 [SMILES: Cc1ccc(F)cc1NC(=O)N[C@H]1CC[C@H](Oc2ccc(C(F)(F)F)cc2)C1]

Compound B-V (61 mg, 0.25 mmol) was dissolved in anhydrous ethyl acetate (4 mL), and a solution of 4-fluoro-2-isocyanato-1-methylbenzene (40 mg, 0.26 mmol) in anhydrous ethyl acetate (3.5 mL) was added dropwise at 55-60° C. The reaction solution was stirred at 60° C. for 2 h, then concentrated in vacuo. The resulting solid residue was recrystallized from hot ethyl acetate to afford the title compound 7-V as white crystalline solid (58 mg, 59%); mp 209.8-211.4° C. (EtOAc). ¹H NMR (400 MHz, DMSO-d₆) δ7.84 (dd, J=12.5, 2.7 Hz, 1H), 7.63 (d, J=2.6 Hz, 1H), 7.61 (d, J=8.8 Hz, 2H), 7.11-7.07 (m, 1H), 7.06 (d, J=8.6 Hz, 2H), 6.87 (d, J=6.9 Hz, 1H), 6.62 (td, J=8.3, 2.8 Hz, 1H), 5.03-4.91 (m, 1H), 4.24-4.06 (m, 1H), 2.22 (ddd, J=20.3, 8.9, 6.2 Hz, 1H), 2.12 (s, 3H), 2.10-2.01 (m, 2H), 1.88-1.78 (m, 1H), 1.78-1.67 (m, 1H), 1.47 (dt, J=16.0, 7.1 Hz, 1H). ¹³C NMR (100 MHz, DMSO-d₆) δ161.07 (d, J=237.5 Hz), 160.75 (d, J=1.1 Hz), 155.09, 140.11 (d, J=11.7 Hz), 131.31 (d, J=9.5 Hz), 127.36 (q, J=3.8 Hz), 125.03 (q, J=271.0 Hz), 121.31 (q, J=32.0 Hz), 121.30 (d, J=2.9 Hz), 116.14, 107.66 (d, J=21.1 Hz), 105.94 (d, J=27.3 Hz), 78.11, 49.86, 31.28, 30.82, 17.59. ¹⁹F NMR (376 MHz, DMSO-d₆) δ−59.83, −116.01 (m). LC-MS (ESI): m/z 397.35 [M+H]⁺, 438.42 [M+H+41]⁺.

Example 3. 1-(3-Cyano-5-(trifluoromethyl)phenyl)-3-(trans-3-(4-(trifluoromethyl) phenoxy)cyclobutyl)urea (1-IV)

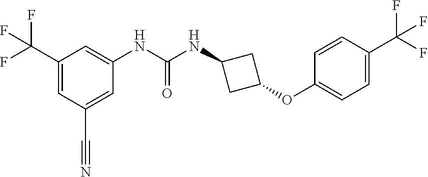 [SMILES: N#Cc1cc(NC(=O)N[C@H]2C[C@H](Oc3ccc(C(F)(F)F)cc3)C2)cc(C(F)(F)F)c1]

The title compound was prepared according to the procedures described in Example 1. White solid (60 mg, 27%); mp 191.6-193.0° C. (EtOAc). ¹H NMR (400 MHz, DMSO-d₆) δ9.09 (s, 1H), 8.15 (s, 1H), 8.03 (s, 1H), 7.77 (s, 1H), 7.62 (d, J=8.7 Hz, 2H), 7.02 (d, J=7.0 Hz, 1H), 6.99 (d, J=8.7 Hz, 2H), 4.99-4.85 (m, 1H), 4.40-4.21 (m, 1H), 2.55-2.35 (m, 4H). ¹³C NMR (100 MHz, DMSO-d₆) δ160.49 (d, J=1.2 Hz), 154.62, 142.67, 131.13 (q, J=32.0 Hz), 127.45 (d, J=3.7 Hz), 125.00 (q, J=270.0 Hz), 124.58, 123.57 (q, J=272.0 Hz), 121.63 (q, J=32.0 Hz), 121.30 (m), 118.60 (m), 118.06, 115.76, 113.27, 70.13, 41.91, 36.93. ¹⁹F NMR (376 MHz, DMSO-d₆) δ−59.88, −61.91. LC-MS (ESI): m/z 444.05 [M+H]⁺, 485.12 [M+H+41]⁺.

Example 4. 1-(trans-3-(4-(Trifluoromethyl)phenoxy)cyclobutyl)-3-(3-(trifluoromethyl) phenyl)urea (2-IV)

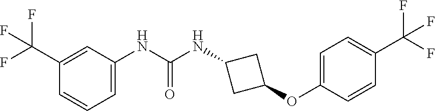 [SMILES: O=C(Nc1cccc(C(F)(F)F)c1)N[C@H]1C[C@H](Oc2ccc(C(F)(F)F)cc2)C1]

The title compound was prepared according to the procedures described in Example 2. White solid (45 mg, 22%); mp 171.8-174.6° C. (EtOAc). ¹H NMR (400 MHz, DMSO-d₆) δ8.78 (s, 1H), 7.94 (s, 1H), 7.63 (d, J=8.6 Hz, 2H), 7.51 (d, J=8.5 Hz, 1H), 7.43 (t, J=7.9 Hz, 1H), 7.21 (d, J=7.6 Hz, 1H), 7.00 (d, J=8.6 Hz, 2H), 6.74 (d, J=7.0 Hz, 1H), 4.97-4.86 (m, 1H), 4.40-4.19 (m, 1H), 2.49-2.28 (m, 4H). ¹³C NMR (100 MHz, DMSO-d₆) δ160.52 (d, J=1.1 Hz), 154.93, 141.63, 130.14, 129.83 (q, J=31.2 Hz), 127.46 (q, J=3.7 Hz), 125.00 (q, J=270.0 Hz), 121.79 (d, J=1.0 Hz), 121.60 (q, J=32.2 Hz), 117.80 (q, J=3.8 Hz), 115.78, 114.17 (q, J=4.0 Hz), 70.17, 41.81, 37.08. ¹⁹F NMR (376 MHz, DMSO-d₆) δ−59.85, −61.35. LC-MS (ESI): m/z 419.11 [M+H]⁺, 460.19 [M+H+41]⁺.

Example 5. 1-(3-Cyanophenyl)-3-(trans-3-(4-(trifluoromethyl) phenoxy)cyclobutyl)urea (3-IV)

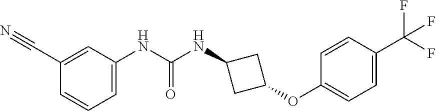 [SMILES: N#Cc1cccc(NC(=O)N[C@H]2C[C@H](Oc3ccc(C(F)(F)F)cc3)C2)c1]

The title compound was prepared according to the procedures described in Example 2. Off white solid (75 mg, 40%); mp 181.1-182.3° C. (EtOAc). ¹H NMR (400 MHz, DMSO-d₆) δ8.77 (s, 1H), 7.90 (s, 1H), 7.63 (d, J=8.7 Hz, 1H), 7.59 (dd, J=8.5, 0.9 Hz, 1H), 7.41 (t, J=7.9 Hz, 1H), 7.32 (d, J=7.6 Hz, 1H), 7.00 (d, J=8.7 Hz, 2H), 6.80 (d, J=7.0 Hz, 1H), 4.99-4.84 (m, 1H), 4.29 (m, 1H), 2.48-2.27 (m, 4H). ¹³C NMR (100 MHz, DMSO-d₆) δ160.51 (d, J=1.1 Hz), 154.82, 141.68, 130.46, 127.46 (q, J=3.8 Hz), 125.06, 125.00 (q, J=270.0 Hz), 122.86, 121.61 (q, J =32.0 Hz), 120.75, 119.39, 115.78, 111.87, 70.16, 41.80, 37.07. ¹⁹F NMR (376 MHz, DMSO-d₆) δ−59.84. LC-MS (ESI): m/z 376.18 [M+H]⁺, 417.19 [M+H+41]⁺.

Example 6. 1-(3-Cyano-5-fluorophenyl)-3-(trans-3-(4-(trifluoromethyl) phenoxy)cyclobutyl)urea (4-IV)

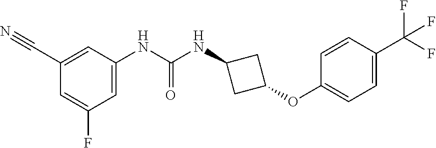 [SMILES: N#Cc1cc(F)cc(NC(=O)N[C@H]2C[C@H](Oc3ccc(C(F)(F)F)cc3)C2)c1]

The title compound was prepared according to the procedures described in Example 1. White crystals (110 mg, 56%); mp 182.8-185.0° C. (EtOAc). ¹H NMR (400 MHz, DMSO-d₆) δ8.96 (s, 1H), 7.65 (d, J=13.3 Hz, 1H), 7.62 (d, J=8.9 Hz, 2H), 7.29 (d, J=7.5 Hz, 1H), 6.99 (d, J=8.5 Hz, 2H), 6.94 (d, J=6.9 Hz, 1H), 4.96-4.86 (m, 1H), 4.34-4.23 (m, 1H), 2.42 (ddd, J=16.3, 12.5, 5.0 Hz, 6H). ¹³C NMR (100 MHz, DMSO-d₆) δ162.38 (d, J=243.6 Hz), 160.49 (d, J=0.9 Hz), 154.55, 143.73 (d, J=12.1 Hz), 127.44 (q, J=3.7 Hz), 124.99 (q, J=271.0 Hz), 121.63 (q, J =32.1 Hz), 118.28 (d, J=4.0 Hz), 117.55 (d, J=2.7 Hz), 115.76, 113.03 (d, J=12.3 Hz), 111.57 (d, J=25.6 Hz), 109.72 (d, J=26.6 Hz), 70.13, 41.84, 36.98. ¹⁹F NMR (376 MHz, DMSO-d₆) δ−59.87, −109.69 (dd, J=11.6, 8.4 Hz). LC-MS (ESI): m/z 394.37 [M+H]⁺, 435.32 [M+H+41]⁺.

Example 7. 1-(5-Fluoro-2-methylphenyl)-3-(trans-3-(4-(trifluoromethyl) phenoxy)cyclobutyl)urea (7-IV)

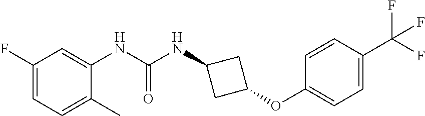 [SMILES: Cc1ccc(F)cc1NC(=O)N[C@H]1C[C@H](Oc2ccc(C(F)(F)F)cc2)C1]

The title compound was prepared according to the procedures described in Example 2. White crystals (130 mg, 68%); mp 204.2-206.0° C. (EtOAc). ¹H NMR (400 MHz, DMSO-d₆) δ7.81 (dd, J=12.4, 2.7 Hz, 1H), 7.70 (s, 1H), 7.63 (d, J=8.6 Hz, 2H), 7.16 (d, J=6.8 Hz, 1H), 7.13-7.07 (m, 1H), 7.00 (d, J=8.6 Hz, 2H), 6.64 (td, J=8.3, 2.8 Hz, 1H), 4.92 (p, J=5.2 Hz, 1H), 4.35-4.22 (m, 1H), 2.46-2.38 (m, 4H), 2.14 (s, 3H). ¹³C NMR (100 MHz, DMSO-d₆) δ161.04 (d, J=237.7 Hz), 160.50 (d, J=1.0 Hz), 154.85 (s), 139.95 (d, J=11.6 Hz), 131.36 (d, J=9.4 Hz), 127.45 (q, J=3.7 Hz), 125.00 (q, J=271.1 Hz), 121.63 (d, J=32.2 Hz), 121.62 (m), 115.79, 107.90 (d, J=21.2 Hz), 106.21 (d, J=27.4 Hz), 70.20, 41.66, 37.20, 17.58. ¹⁹F NMR (376 MHz, DMSO-d₆) δ−59.86, −116.04 (m). LC-MS (ESI): m/z 383.32 [M+H]⁺, 424.40 [M+H+41]⁺.

Example 8. 1-(3,4-difluorophenyl)-3-(trans-3-(4-(trifluoromethyl) phenoxy)cyclobutyl)urea (10-IV)

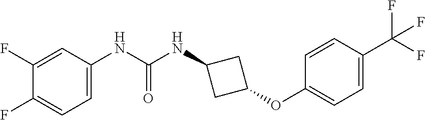 [SMILES: O=C(Nc1ccc(F)c(F)c1)N[C@H]1C[C@H](Oc2ccc(C(F)(F)F)cc2)C1]

The title compound was prepared according to the procedures described in Example 2. White solid (63 mg, 33%); mp 179.9-182.0° C. ¹H NMR (400 MHz, DMSO-d₆) δ8.62 (s, 1H), 7.62 (d, J=8.6 Hz, 2H), 7.61-7.55 (m, 1H), 7.25 (dd, J=19.7, 9.3 Hz, 1H), 7.05-7.01 (m, 1H), 6.99 (d, J=8.6 Hz, 2H), 6.68 (d, J=7.0 Hz, 1H), 4.97-4.84 (m, 1H), 4.37-4.19 (m, 1H), 2.41 (m, 4H). ¹³C NMR (100 MHz, DMSO-d₆) δ160.52 (d, J=1.3 Hz), 154.89, 149.47 (dd, J=242.0, 13.3 Hz), 144.45 (dd, J=238.6, 12.7 Hz), 137.97 (dd, J=9.5, 2.5 Hz), 127.45 (q, J=3.8 Hz), 125.00 (q, J=271.0 Hz), 121.60 (q, J=32.1 Hz), 117.59 (d, J=18.6 Hz), 115.77, 114.23 (dd, J=5.7, 3.2 Hz), 107.07 (d, J=21.8 Hz), 70.16, 41.78, 37.11. ¹⁹F NMR (376 MHz, DMSO-d₆) δ−59.86, −137.83 (ddd, J=23.1, 13.5, 9.4 Hz), −148.15 (m). LC-MS (ESI): m/z 387.22 [M-H]⁺, 428.24 [M+H+41]⁺.

Example 9. 1-(2,3-Difluorophenyl)-3-(trans-3-(4-(trifluoromethyl) phenoxy)cyclobutyl)urea (11-IV)

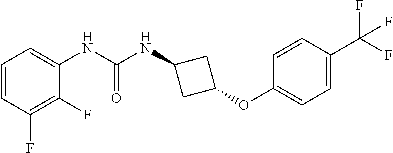 [SMILES: O=C(Nc1cccc(F)c1F)N[C@H]1C[C@H](Oc2ccc(C(F)(F)F)cc2)C1]

The title compound was prepared according to the procedures described in Example 2. White crystals (120 mg, 69%); mp 205.9-208.0° C. (EtOAc-heptane). ¹H NMR (400 MHz, DMSO-d₆) δ8.83 (s, 1H), 7.62 (d, J=8.6 Hz, 2H), 7.11 (m, 1H), 6.99 (d, J=8.6 Hz, 2H), 6.80 (d, J=6.9 Hz, 1H), 6.73-6.59 (m, 1H), 5.00-4.82 (m, 1H), 4.42-4.11 (m, 1H), 2.41 (m, 4H). ¹³C NMR (100 MHz, DMSO-d₆) δ163.08 (d, J=241.8 Hz), 162.92 (d, J=241.8 Hz), 160.50, 154.61, 143.47 (d, J=13.8 Hz), 127.44 (q, J=3.7 Hz), 124.99 (d, J=270.9 Hz), 121.61 (d, J=32.1 Hz), 115.76, 101.01, 100.71, 96.30 (d, J=27.4 Hz), 70.14, 41.77, 37.03. ¹⁹F NMR (376 MHz, DMSO-d₆) δ−59.87, −110.04 (d, J=9.4 Hz), −110.06 (d, J=9.4 Hz). LC-MS (ESI): m/z 387.29 [M+H]⁺, 428.37[M+H+41]⁺.

Example 10. 1-(3-Cyano-5-(trifluoromethyl)phenyl)-3-(cis-3-(4-(trifluoromethyl) phenoxy)cyclobutyl)urea (1-IVc)

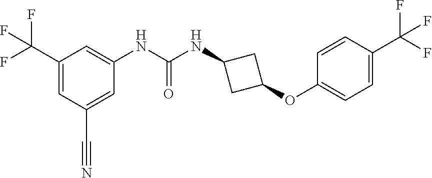 [SMILES: N#Cc1cc(NC(=O)N[C@H]2C[C@@H](Oc3ccc(C(F)(F)F)cc3)C2)cc(C(F)(F)F)c1]

The title compound was prepared according to the procedures described in Example 1. White solid (80 mg, 62%); mp 194.6-196.1° C. (EtOAc). ¹H NMR (400 MHz, DMSO-d₆) δ9.08 (s, 1H), 8.13 (s, 1H), 8.03 (s, 1H), 7.77 (s, 1H), 7.62 (d, J=8.6 Hz, 2H), 7.03 (d, J=8.6 Hz, 2H), 6.94 (d, J=7.8 Hz, 1H), 4.54 (p, J=6.9 Hz, 1H), 4.02-3.81 (m, 1H), 2.87 (m, 2H), 2.05 (td, J=9.1, 2.8 Hz, 2H). ¹³C NMR (100 MHz, DMSO-d₆) δ160.34 (d, J=1.2 Hz), 154.37, 142.67, 131.11 (q, J=33.0 Hz), 127.46 (q, J=3.7 Hz), 124.97 (q, J=270.9 Hz), 124.60, 123.57 (q, J=272.7 Hz), 121.66 (q, J=32.0 Hz), 121.30 (q, J=3.7 Hz), 118.63 (q, J=3.9 Hz), 118.06, 115.67, 113.26, 65.86, 38.69, 37.32. ¹⁹F NMR (376 MHz, DMSO-d₆) δ−59.88, −61.89. LC-MS (ESI): m/z 444.05 [M+H]⁺, 485.12 [M+H+41]⁺.

Example 11. 1-(cis-3-(4-(Trifluoromethyl)phenoxy)cyclobutyl)-3-(3-(trifluoromethyl) phenyl)urea (2-IVc)

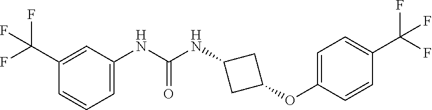 [SMILES: O=C(Nc1cccc(C(F)(F)F)c1)N[C@H]1C[C@@H](Oc2ccc(C(F)(F)F)cc2)C1]

The title compound was prepared according to the procedures described in Example 2. White solid (40 mg, 32%); mp 166.6-168.5° C. ¹H NMR (400 MHz, DMSO-d₆) δ8.84 (s, 1H), 8.01 (s, 1H), 7.70 (d, J=8.7 Hz, 2H), 7.58 (d, J=8.5 Hz, 1H), 7.50 (t, J=7.9 Hz, 1H), 7.29 (d, J=7.6 Hz, 1H), 7.12 (d, J=8.6 Hz, 2H), 6.73 (d, J=7.9 Hz, 1H), 4.62 (p, J=7.1 Hz, 1H), 4.09-3.91 (m, 1H), 3.06-2.85 (m, 2H), 2.20-2.01 (m, 2H). ¹³C NMR (100 MHz, DMSO-d₆) δ160.37 (d, J=1.1 Hz), 154.66, 141.62, 130.14, 129.81 (d, J=31.2 Hz), 127.46 (q, J=3.7 Hz), 124.98 (d, J =270.9 Hz), 124.70 (d, J=272.3 Hz), 121.81 (d, J=1.2 Hz), 121.64 (d, J=32.5 Hz), 117.81 (q, J=7.6, 4.0 Hz), 115.69, 114.19 (q, J=7.9, 4.1 Hz), 65.87, 38.88, 37.22. ¹⁹F NMR (376 MHz, DMSO-d₆) δ−59.86, −61.35. LC-MS (ESI): m/z 418.98 [M+H]⁺, 460.05 [M+H+41]⁺.

Example 12. 1-(3-Cyanophenyl)-3-(cis-3-(4-(trifluoromethyl) phenoxy)cyclobutyl)urea (3-IVc)

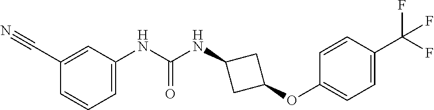 [SMILES: N#Cc1cccc(NC(=O)N[C@H]2C[C@@H](Oc3ccc(C(F)(F)F)cc3)C2)c1]

The title compound was prepared according to the procedures described in Example 20. Off white solid (180 mg, 96%); mp 203.7-206.8° C. ¹H NMR (400 MHz, DMSO-d₆) δ8.74 (s, 1H), 7.90 (t, J=1.7 Hz, 1H), 7.62 (d, J=8.7 Hz, 2H), 7.60-7.56 (m, 1H), 7.40 (t, J=7.9 Hz, 1H), 7.31 (m, 1H), 7.03 (d, J=8.6 Hz, 2H), 6.70 (d, J=7.9 Hz, 1H), 4.53 (p, J=7.0 Hz, 1H), 4.02-3.83 (m, 1H), 2.97-2.77 (m, 2H), 2.01 (m, 2H). ¹³C NMR (100 MHz, DMSO-d₆) δ160.36 (d, J=1.1 Hz), 154.56, 141.68, 130.44, 127.45 (q, J=3.7 Hz), 125.06, 124.98 (q, J=271.0 Hz), 122.88, 121.65 (q, J=32.0 Hz), 120.78, 119.39, 115.67, 111.87, 65.86, 38.87, 37.21. ¹⁹F NMR (376 MHz, DMSO-d₆) δ−59.87. LC-MS (ESI): m/z 376.11 [M+H]⁺, 417.12 [M+H+41]⁺.

Example 13. 1-(3-Cyano-5-(trifluoromethyl)phenyl)-3-((1S,3S)-3-(4-(trifluoromethyl) phenoxy)cyclopentyl)urea (1-V)

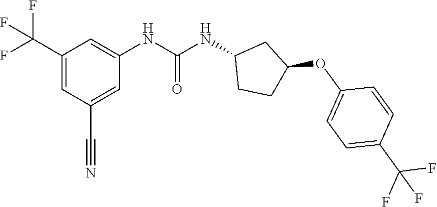 [SMILES: N#Cc1cc(NC(=O)N[C@H]2CC[C@H](Oc3ccc(C(F)(F)F)cc3)C2)cc(C(F)(F)F)c1]

The title compound was prepared according to the procedures described in Example 1. White solid (80 mg, 70%); mp 144.4-146.2° C. (EtOAc-heptane). ¹H NMR (400 MHz, DMSO-d₆) δ8.99 (s, 1H), 8.14 (s, 1H), 8.00 (s, 1H), 7.76 (s, 1H), 7.61 (d, J=8.7 Hz, 2H), 7.06 (d, J=8.6 Hz, 2H), 6.69 (d, J=7.2 Hz, 1H), 5.04-4.90 (m, 1H), 4.29-4.05 (m, 1H), 2.33-2.11 (m, 1H), 2.06 (dt, J=13.3, 7.0 Hz, 2H), 1.96-1.80 (m, 1H), 1.80-1.62 (m, 1H), 1.51 (dt, J=16.1, 7.3 Hz, 1H). ¹³C NMR (100 MHz, DMSO-d₆) δ160.73 (d, J=1.1 Hz), 154.83, 142.73, 131.14 (q, J=32.8 Hz), 127.37 (d, J=3.8 Hz), 125.02 (d, J=270.8 Hz), 124.43, 123.58 (q, J=272.9 Hz), 121.63 (q, J=31.9 Hz), 121.17 (q, J=2.7 Hz), 118.44 (q, J=3.9 Hz), 118.06, 116.13, 113.28, 78.11, 50.01, 31.01, 30.78. ¹⁹F NMR (376 MHz, DMSO-d₆) δ−59.83, −61.91. LC-MS (ESI): m/z 458.33 [M+H]⁺, 499.34 [M+H+41]⁺.

Example 14. 14(1S,3S)-3-(4-(Trifluoromethyl)phenoxy)cyclopentyl)-3-(3-(trifluoromethyl) phenyl)urea (2-V)

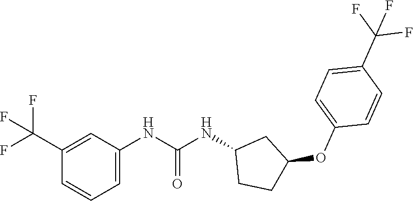 [SMILES: O=C(Nc1cccc(C(F)(F)F)c1)N[C@H]1CC[C@H](Oc2ccc(C(F)(F)F)cc2)C1]

The title compound was prepared according to the procedures described in Example 2. Off white solid (60 mg, 56%); mp 123.5-125.3° C. (EtOAc-heptane). ¹H NMR (400 MHz, DMSO-d₆) δ8.68 (s, 1H), 7.95 (s, 1H), 7.61 (d, J=8.7 Hz, 2H), 7.51-7.37 (m, 2H), 7.19 (d, J=7.4 Hz, 1H), 7.06 (d, J=8.7 Hz, 2H), 6.40 (d, J=7.2 Hz, 1H), 5.02-4.90 (m, 1H), 4.25-4.07 (m, 1H), 2.21 (ddd, J=20.3, 9.0, 6.1 Hz, 1H), 2.14-1.98 (m, 2H), 1.92-1.79 (m, 1H), 1.79-1.63 (m, 1H), 1.49 (m, 1H). ¹³C NMR (100 MHz, DMSO-d₆) δ160.75 (d, J=1.4 Hz), 155.15, 141.71, 130.13, 129.85 (q, J=31.3 Hz), 127.37 (q, J=3.8 Hz), 125.03 (d, J=270.7 Hz), 124.70 (q, J=272.2 Hz),121.61, 121.30 (q, J=32.1 Hz), 117.66 (q, J=3.7 Hz), 116.13, 113.99 (q, J=4.1 Hz), 78.13, 49.90, 31.15, 30.79. ¹⁹F NMR (376 MHz, DMSO-d₆) δ−59.82, −61.38. LC-MS (ESI): m/z 433.13 [M+H]⁺, 474.21 [M+H+41]⁺.

Example 15. 1-(3-Cyanophenyl)-34(1S,3S)-3-(4-(trifluoromethyl) phenoxy)cyclopentyl)urea (3-V)

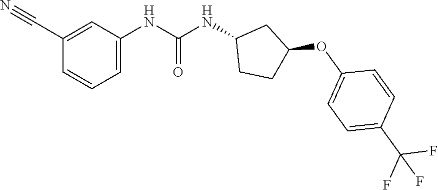 [SMILES: N#Cc1cccc(NC(=O)N[C@H]2CC[C@H](Oc3ccc(C(F)(F)F)cc3)C2)c1]

The title compound was prepared according to the procedures described in Example 2. White solid (65 mg, 67%); mp 160.2-162.1° C. (EtOAc-heptane). ¹H NMR (400 MHz, DMSO-d₆) δ8.66 (s, 1H), 7.90 (t, J=1.7 Hz, 1H), 7.61 (d, J=8.7 Hz, 2H), 7.56 (dd, J=8.3, 1.1 Hz, 1H), 7.40 (t, J=7.9 Hz, 1H), 7.34-7.27 (m, 1H), 7.06 (d, J=8.6 Hz, 2H), 6.46 (d, J=7.2 Hz, 1H), 5.02-4.90 (m, 1H), 4.24-4.07 (m, 1H), 2.21 (ddd, J=20.2, 9.0, 6.1 Hz, 1H), 2.06 (ddd, J=20.4, 13.8, 7.0 Hz, 2H), 1.85 (ddd, J=14.1, 7.9, 6.4 Hz, 1H), 1.79-1.63 (m, 1H), 1.55-1.40 (m, 1H). ¹³C NMR (100 MHz, DMSO-d₆) δ160.75 (d, J=1.2 Hz), 155.04, 141.77, 130.45, 127.37 (q, J=3.8 Hz), 125.03 (q, J=270.9 Hz), 124.92, 122.68, 121.30 (q, J=32.1 Hz), 120.57, 119.40, 116.13, 111.89, 78.12, 49.91, 31.15, 30.79. ¹⁹F NMR (376 MHz, DMSO-d₆) δ−59.82. LC-MS (ESI): m/z 390.33 [M+H]⁺, 431.35 [M+H+41]⁺.

Example 16. 1-(3-Cyano-5-fluorophenyl)-3-((1S,3S)-3-(4-(trifluoromethyl) phenoxy)cyclopentypurea (4-V)

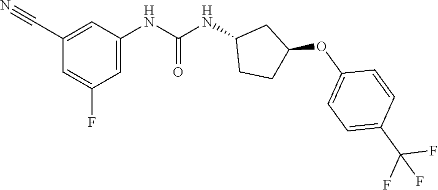 [SMILES: N#Cc1cc(F)cc(NC(=O)N[C@H]2CC[C@H](Oc3ccc(C(F)(F)F)cc3)C2)c1]

The title compound was prepared according to the procedures described in Example 1. Off white solid (50 mg, 49%); mp 176.2-177.8° C. (EtOAc-heptane). ¹H NMR (400 MHz, DMSO-d₆) δ8.86 (s, 1H), 7.65 (s, 1H), 7.61 (d, J=8.9 Hz, 2H), 7.61 (d, J=13.9 Hz, 1H), 7.33-7.22 (m, 1H), 7.06 (d, J=8.6 Hz, 2H), 6.61 (d, J=7.2 Hz, 1H), 5.04-4.88 (m, 1H), 4.26-4.07 (m, 1H), 2.21 (m, 1H), 2.06 (m, 2H), 1.93-1.79 (m, 1H), 1.71 (m, 1H), 1.49 (m, 1H). ¹³C NMR (100 MHz, DMSO-d₆) δ162.40 (d, J=243.3 Hz), 160.73 (d, J=1.2 Hz), 154.77, 143.80 (d, J=12.2 Hz), 127.37 (q, J=3.8 Hz), 125.02 (d, J=271.0 Hz), 121.31 (d, J=32.1 Hz), 118.29 (d, J =3.7 Hz), 117.40 (d, J=2.6 Hz), 116.13, 113.04 (d, J=12.3 Hz), 111.45 (d, J=25.6 Hz), 109.57 (d, J=26.2 Hz), 78.10, 49.95, 31.06, 30.78. ¹⁹F NMR (376 MHz, DMSO-d₆) δ−59.82, −109.71 (dd, J=11.7, 8.4 Hz). LC-MS (ESI): m/z 408.1 [M+H]⁺, 449.1 [M+H+41]⁺.

Example 17. 1-(3,4-Difluorophenyl)-3-((1S,3S)-3-(4-(trifluoromethyl) phenoxy)cyclopentyl)urea (10-V)

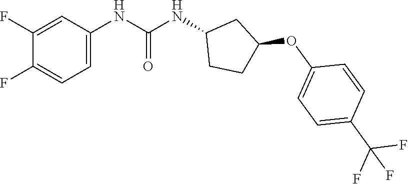 [SMILES: O=C(Nc1ccc(F)c(F)c1)N[C@H]1CC[C@H](Oc2ccc(C(F)(F)F)cc2)C1]

The title compound was prepared according to the procedures described in Example 2. White solid (40 mg, 34%); mp 175.4-177.5° C. ¹H NMR (400 MHz, DMSO-d₆) δ8.52 (s, 1H), 7.61 (d, J=8.9 Hz, 2H), 7.58 (dd, J=7.6, 2.6 Hz, 1H), 7.24 (dd, J=19.8, 9.2 Hz, 1H), 7.06 (d, J=8.6 Hz, 2H), 7.02-6.96 (m, 1H), 6.34 (d, J=7.2 Hz, 1H), 5.01-4.89 (m, 1H), 4.23-4.06 (m, 1H), 2.20 (ddd, J=20.3, 8.9, 6.0 Hz, 1H), 2.05 (ddd, J=20.1, 13.6, 6.8 Hz, 2H), 1.90-1.77 (m, 1H), 1.77-1.62 (m, 1H), 1.47 (dt, J=16.1, 7.3 Hz, 1H). ¹³C NMR (100 MHz, DMSO-d₆) δ160.74, 155.12, 149.49 (dd, J=241.8, 13.0 Hz), 144.36 (dd, J=238.6, 12.8 Hz), 138.07 (dd, J=9.5, 2.5 Hz), 127.36 (q, J=3.7 Hz), 125.03 (q, J=270.8 Hz), 121.29 (q, J=32.4 Hz), 117.60 (d, J=17.2 Hz), 116.13, 114.02 (dd, J=5.7, 3.1 Hz), 106.88 (d, J=21.8 Hz), 78.11, 49.86, 31.19, 30.78. ¹⁹F NMR (376 MHz, DMSO-d₆) δ−59.82, −137.83 (ddd, J=23.1, 13.6, 9.5 Hz), −148.33 (dddd, J=22.5, 11.1, 7.5, 3.9 Hz). LC-MS (ESI): m/z 401.31 [M+H]⁺, 442.39 [M+H+41]⁺.

Example 18. 1-(4-Cyano-3-fluorophenyl)-3-(trans-4-(4-(trifluoromethyl) phenoxy)cyclohexyl)urea (5-VI)

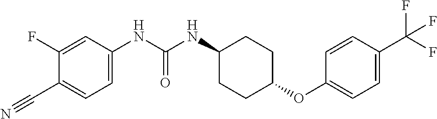 [SMILES: N#Cc1ccc(NC(=O)N[C@H]2CC[C@H](Oc3ccc(C(F)(F)F)cc3)CC2)cc1F]

The title compound was prepared according to the procedures described in

Example 1. White crystalline solid (100 mg, 47%); mp 226.0-228.0° C. (EtOAc). ¹H NMR (400 MHz, DMSO-d₆) δ9.08 (s, 1H), 7.73-7.54 (m, 4H), 7.19-7.06 (m, 3H), 6.49 (d, J=7.5 Hz, 1H), 4.45 (ddd, J=13.5, 9.5, 3.8 Hz, 1H), 3.59-3.47 (m, 1H), 2.04 (d, J=10.0 Hz, 2H), 2.01-1.79 (m, 2H), 1.62-1.26 (m, 4H). ¹H NMR (400 MHz, DMSO-d₆) δ9.08 (s, 1H), 7.68 (t, J=8.2 Hz, 1H), 7.65 (d, J=1.9 Hz, 1H), 7.59 (d, J=8.7 Hz, 2H), 7.15 (dd, J=8.7, 1.8 Hz, 1H), 7.11 (d, J=8.6 Hz, 2H), 6.49 (d, J=7.5 Hz, 1H), 4.45 (ddd, J=13.5, 9.5, 3.8 Hz, 1H), 3.59-3.48 (m, 1H), 2.04 (d, J=10.0 Hz, 2H), 2.01-1.79 (m, 2H), 1.62-1.26 (m, 4H). ¹³C NMR (100 MHz, DMSO-d₆) δ163.72 (d, J=251.2 Hz), 160.71 (d, J=1.2 Hz), 154.10, 147.68 (d, J=12.1 Hz), 134.42 (d, J=1.9 Hz), 127.36 (q, J=3.6 Hz), 125.01 (q, J=270.8 Hz), 121.27 (q, J=32.0 Hz), 116.26, 115.12, 114.18 (d, J=2.1 Hz), 104.25 (d, J=24.9 Hz), 91.14 (d, J=15.6 Hz), 74.79, 47.73, 30.11, 29.89. ¹⁹F NMR (376 MHz, DMSO-d₆) δ−59.83, −107.34 (dd, J=12.8, 7.8 Hz). LC-MS (ESI): m/z 421.95[M+H]⁺, 463.03 [M+H+41]⁺.

Example 19. 1-(5-Cyano-2-fluorophenyl)-3-(trans-4-(4-(trifluoromethyl) phenoxy)cyclohexyl)urea (6-VI)

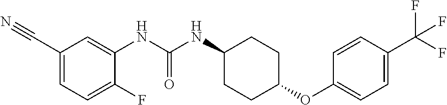 [SMILES: N#Cc1ccc(F)c(NC(=O)N[C@H]2CC[C@H](Oc3ccc(C(F)(F)F)cc3)CC2)c1]

The title compound was prepared according to the procedures described in Example 1. Off white solid (110 mg, 52%); mp 234.6-237.0° C. (EtOAc). ¹H NMR (400 MHz, DMSO-d₆) δ8.56 (d, J=7.8 Hz, 1H), 8.53 (d, J=2.8 Hz, 1H), 7.60 (d, J =8.7 Hz, 2H), 7.45-7.36 (m, 2H), 7.12 (d, J=8.6 Hz, 2H), 6.77 (d, J=7.4 Hz, 1H), 4.48 (ddd, J=13.4, 9.4, 3.7 Hz, 1H), 3.65-3.39 (m, 1H), 2.12-1.98 (m, 2H), 1.99-1.87 (m, 2H), 1.51 (td, J=12.5, 2.9 Hz, 2H), 1.36 (td, J=12.7, 2.8 Hz, 2H). ¹³C NMR (100 MHz, DMSO-d₆) δ160.72 (d, J=1.1 Hz), 154.26, 154.01 (d, J=250.5 Hz), 130.18 (d, J=11.5 Hz), 127.36 (q, J=3.6 Hz), 126.20 (d, J=8.6 Hz), 125.01 (q, J=270.8 Hz), 122.92 (d, J=3.8 Hz), 121.27 (q, J=31.9 Hz), 118.90, 116.94 (d, J=20.9 Hz), 116.28, 107.99 (d, J=3.4 Hz), 74.64, 47.54, 30.05, 29.68. ¹⁹F NMR (376 MHz, DMSO-d₆) δ−59.81, −121.67. LC-MS (ESI): m/z 422.02[M+H]⁺, 463.23 [M+H+41]⁺.

Example 20. 1-(5-Fluoro-2-methylphenyl)-3-(trans-4-(4-(trifluoromethyl) phenoxy)cyclohexyl)urea (7-VI)

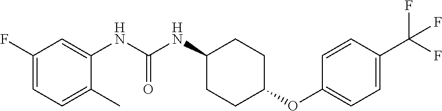 [SMILES: Cc1ccc(F)cc1NC(=O)N[C@H]1CC[C@H](Oc2ccc(C(F)(F)F)cc2)CC1]

The title compound was prepared according to the procedures described in Example 2. White solid (80 mg, 39%); mp 238.2-239.8° C. (EtOAc). ¹N MR (400 MHz, DMSO-d₆) δ7.88 (dd, J=12.6, 2.7 Hz, 1H), 7.65 (s, 1H), 7.60 (d, J=8.7 Hz, 2H), 7.12 (d, J=8.8 Hz, 2H), 7.08 (d, J=7.5 Hz, 1H), 6.77 (d, J=7.4 Hz, 1H), 6.61 (td, J=8.3, 2.8 Hz, 1H), 4.48 (ddd, J=13.4, 9.5, 3.7 Hz, 1H), 3.69-3.47 (m, 1H), 2.13 (s, 2H), 2.12-2.00 (m, 2H), 1.95 (dd, J=12.9, 3.0 Hz, 2H), 1.50 (td, J=12.5, 2.8 Hz, 2H), 1.35 (td, J=12.7, 2.7 Hz, 2H). ¹³C NMR (100 MHz, DMSO-d₆) δ161.08 (d, J=237.4 Hz), 160.75 (d, J=1.2 Hz), 154.80, 140.22 (d, J=11.8 Hz), 131.30 (d, J=9.5 Hz), 127.36 (q, J=3.7 Hz), 125.02 (q, J=270.9 Hz), 121.27 (q, J =32.1 Hz), 121.15 (d, J=2.7 Hz), 116.28, 107.52 (d, J=21.0 Hz), 105.78 (d, J=27.3 Hz), 74.83, 47.59, 30.31, 29.90, 17.64. ¹⁹F NMR (376 MHz, DMSO-d₆) δ−59.81, −115.96 (dd, J=13.2, 6.7 Hz). LC-MS (ESI): m/z 410.97[M+H]⁺, 452.05[M+H+41]⁺.

Example 21. 1-(4-Cyano-2-fluorophenyl)-3-(trans-4-(4-(trifluoromethyl) phenoxy)cyclohexyl)urea (8-VI)

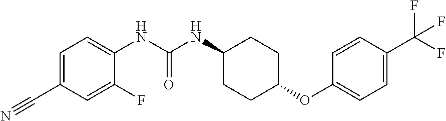 [SMILES: N#Cc1ccc(NC(=O)N[C@H]2CC[C@H](Oc3ccc(C(F)(F)F)cc3)CC2)c(F)c1]

The title compound was prepared according to the procedures described in Example 1. White solid (100 mg, 47%); mp 221.6-223.6° C. (EtOAc). ¹H NMR (400 MHz, DMSO-d₆) δ8.66 (d, J=2.0 Hz, 1H), 8.38 (t, J=8.4 Hz, 1H), 7.77 (dd, J=11.5, 1.8 Hz, 1H), 7.60 (d, J=8.7 Hz, 2H), 7.57-7.50 (m, 1H), 7.11 (d, J=8.7 Hz, 2H), 6.87 (d, J=7.4 Hz, 1H), 4.48 (ddd, J=13.2, 9.3, 3.6 Hz, 1H), 3.61-3.49 (m, 1H), 2.15-1.99 (m, 2H), 1.94 (dd, J=12.9, 3.4 Hz, 2H), 1.61-1.40 (m, 2H), 1.35 (dt, J=12.9, 6.6 Hz, 2H). ¹³C NMR (100 MHz, DMSO-d₆) δ160.72 (d, J=1.1 Hz), 153.90, 150.46 (d, J=243.0 Hz), 134.09 (d, J=10.0 Hz), 130.12 (d, J=3.2 Hz), 127.36 (q, J=3.8 Hz), 125.01 (d, J=271.0 Hz), 121.27 (d, J=32.0 Hz), 119.44 (d, J =2.8 Hz), 118.99 (d, J=22.9 Hz), 118.77 (d, J=2.6 Hz), 116.27, 102.60 (d, J=9.5 Hz), 74.62, 47.52, 30.00, 29.65. ¹⁹F NMR (376 MHz, DMSO-d₆) δ−59.82, −129.13. LC-MS (ESI): m/z 422.1 [M+H]⁺, 463.1 [M+H+41]⁺.

Example 22. 1-(3-Cyano-5-fluorophenyl)-3-(trans-4-(4-(trifluoromethyl) phenoxy)cyclohexyl)urea (9-VI)

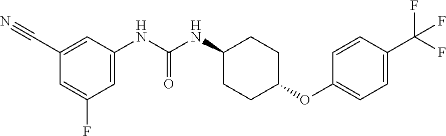 [SMILES: N#Cc1cc(F)cc(NC(=O)N[C@H]2CC[C@H](Oc3ccc(C(F)(F)F)cc3)CC2)c1]

The title compound was prepared according to the procedures described in Example 1. Pink solid (105 mg, 50%); mp 219.8-222.0° C. (EtOAc). ¹H NMR (400 MHz, DMSO-d₆) δ8.62 (s, 1H), 7.90 (dd, J=5.7, 2.7 Hz, 1H), 7.64-7.57 (m, 1H), 7.59 (d, J=8.7 Hz, 2H), 7.37 (t, J=9.1 Hz, 1H), 7.11 (d, J=8.7 Hz, 2H), 6.32 (d, J =7.6 Hz, 1H), 4.44 (ddd, J=13.6, 9.5, 3.8 Hz, 1H), 3.58-3.46 (m, 1H), 2.09-1.98 (m, 2H), 1.96-1.88 (m, 2H), 1.59-1.25 (m, 4H). ¹³C NMR (100 MHz, DMSO-d₆) δ160.73 (d, J=1.2 Hz), 157.36 (d, J=249.4 Hz), 154.75, 138.14 (d, J=2.7 Hz), 127.36 (q, J=3.6 Hz), 125.18 (d, J=7.7 Hz), 125.01 (q, J=270.9 Hz), 121.33, 121.26 (q, J=32.1 Hz), 117.26 (d, J=20.4 Hz), 116.27, 114.65, 100.09 (d, J=16.0 Hz), 74.86, 47.70, 30.28, 29.96. ¹⁹F NMR (376 MHz, DMSO-d₆) δ−59.82, −118.72 (m). LC-MS (ESI): m/z 422.22 [M-H]⁺, 463.23 [M+H+41]⁺.

Example 23. 1-(3,4-Difluorophenyl)-3-(trans-4-(4-(trifluoromethyl) phenoxy)cyclohexyl)urea (10-VI)

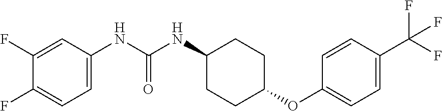 [SMILES: O=C(Nc1ccc(F)c(F)c1)N[C@H]1CC[C@H](Oc2ccc(C(F)(F)F)cc2)CC1]

The title compound was prepared according to the procedures described in Example 2. White solid (68 mg, 33%); mp 228.2-229.8° C. (EtOAc). ¹H NMR (400 MHz, DMSO-d₆) δ8.49 (s, 1H), 7.63-7.57 (m, 1H), 7.60 (d, J=8.7 Hz, 2H), 7.24 (dd, J=19.7, 9.3 Hz, 1H), 7.11 (d, J=8.6 Hz, 2H), 7.03 - 6.92 (m, 1H), 6.19 (d, J=7.6 Hz, 1H), 4.44 (ddd, J=13.6, 9.5, 3.7 Hz, 1H), 3.57-3.46 (m, 1H), 2.16-1.94 (m, 2H), 1.94-1.79 (m, 2H), 1.59-1.25 (m, 4H). ¹³C NMR (100 MHz, DMSO-d₆) δ160.73 (d, J=1.2 Hz), 154.79, 149.51 (dd, J=241.8, 13.1 Hz), 144.32 (dd, J=238.6, 12.8 Hz), 138.10 (dd, J=9.5, 2.5 Hz), 127.36 (q, J=3.8 Hz), 125.01 (q, J=270.7 Hz), 121.26 (q, J=32.1 Hz), 117.62 (d, J=16.8 Hz), 116.26, 113.88 (dd, J=5.7, 3.2 Hz), 106.75 (d, J=21.9 Hz), 74.87, 47.60, 30.33, 29.96. ¹⁹F NMR (376 MHz, DMSO-d₆) δ−59.82, −137.77 (ddd, J=23.1, 13.6, 9.5 Hz), −148.40 (dddd, J=22.4, 11.0, 7.5, 3.8 Hz). LC-MS (ESI): m/z 415.21 [M-H]⁺, 456.22 [M+H+41]⁺.

Example 24. 1-(2,3-Difluorophenyl)-3-(trans-4-(4-(trifluoromethyl) phenoxy)cyclohexyl)urea (11-VI)

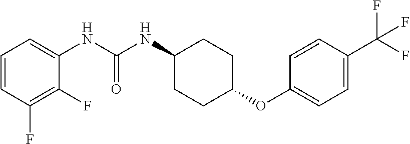 [SMILES: O=C(Nc1cccc(F)c1F)N[C@H]1CC[C@H](Oc2ccc(C(F)(F)F)cc2)CC1]

The title compound was prepared according to the procedures described in Example 2. Off white solid (90 mg, 43%); mp 227.3-229.9° C. (EtOAc). ¹H NMR (400 MHz, DMSO-d₆) δ8.69 (s, 1H), 7.60 (d, J=8.7 Hz, 2H), 7.11 (d, J=8.6 Hz, 2H), 7.10-7.05 (m, 1H), 6.65 (tt, J=9.4, 2.3 Hz, 1H), 6.31 (d, J=7.6 Hz, 1H), 4.52-4.37 (m, 1H), 3.62-3.42 (m, 1H), 2.16-1.94 (m, 2H), 1.91 (dd, J=12.9, 3.2 Hz, 2H), 1.59-1.26 (m, 4H). ¹³C NMR (100 MHz, DMSO-d₆) δ163.04 (dd, J=241.6, 15.8 Hz), 160.73 (d, J=1.0 Hz), 154.49, 127.36 (q, J=3.9 Hz), 125.01 (q, J=270.9 Hz), 121.26 (q, J=32.1 Hz), 120.85 (d, J=6.6 Hz), 116.27, 100.62 (d, J=29.3 Hz), 100.62 (d, J=12.4 Hz), 96.27 (t, J=26.3 Hz), 74.85, 47.65, 30.24, 29.95. ¹⁹F NMR (376 MHz, DMSO-d₆) δ−59.82, −110.01 (d, J=9.5 Hz), −110.03 (d, J=9.5 Hz). LC-MS (ESI): m/z 415.01 [M+H]⁺, 456.08 [M+H+41]⁺.

Example 25. 1-(3-Cyano-5-(trifluoromethyl)phenyl)-3-(5-(4-(trifluoromethyl) phenoxy)pyrimidin-2-yl)urea (1-py)

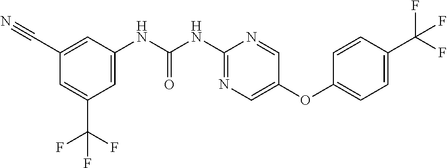 [SMILES: N#Cc1cc(NC(=O)Nc2ncc(Oc3ccc(C(F)(F)F)cc3)cn2)cc(C(F)(F)F)c1]

The title compound was prepared according to the procedures described in Example 1. White solid (30 mg, 13%); mp 263.5-265.5° C. (EtOAc). ¹H NMR (400 MHz, DMSO-d₆) δ11.83 (s, 1H), 10.65 (s, 1H), 8.66 (d, J=3.0 Hz, 2H), 8.38 (d, J=13.9 Hz, 2H), 7.96 (s, 1H), 7.76 (d, J=6.5 Hz, 2H), 7.30 (d, J=6.3 Hz, 2H). ¹³C NMR (100 MHz, DMSO-d₆) δ160.59 (d, J=0.8 Hz), 154.70, 151.99, 151.13, 145.44, 140.98, 131.34 (q, J=32.7 Hz), 128.09 (q, J=3.9 Hz), 126.45, 124.60 (q, J=271.4 Hz), 124.55 (q, J=31.9 Hz), 124.53 (q, J=269.1 Hz), 123.24 (d, J=4.4 Hz), 120.22 (d, J=4.3 Hz), 118.18, 117.87, 113.50. ¹⁹F NMR (376 MHz, DMSO-d₆) δ−60.31, −61.68. LC-MS (ESI): m/z 467.99 [M+H]⁺, 509.06 [M+H+41]⁺.

Example 26. 1-(5-(4-(Trifluoromethyl)phenoxy)pyrimidin-2-yl)-3-(3-(trifluoromethyl) phenyl)urea (2-py)

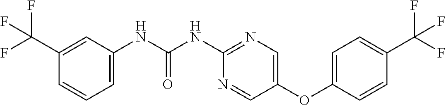 [SMILES: O=C(Nc1cccc(C(F)(F)F)c1)Nc1ncc(Oc2ccc(C(F)(F)F)cc2)cn1]

The title compound was prepared according to the procedures described in Example 2. White solid (55 mg, 31%); mp 232.5-234.8° C. (EtOAc). ¹H NMR (400 MHz, DMSO-d₆) δ11.49 (s, 1H), 10.45 (s, 1H), 8.67 (s, 2H), 8.07 (s, 1H), 7.82 (d, J=8.2 Hz, 1H), 7.75 (d, J=8.7 Hz, 2H), 7.55 (t, J=8.0 Hz, 1H), 7.38 (d, J=7.7 Hz, 1H), 7.29 (d, J=8.6 Hz, 2H). ¹³C NMR (100 MHz, DMSO-d₆) δ160.75 (d, J=1.2 Hz), 155.01, 151.89, 151.35, 145.11, 139.89, 130.44, 130.08 (d, J=31.5 Hz), 128.05 (q, J=3.7 Hz), 124.62 (q, J=271.4 Hz), 124.57 (q, J=272.2 Hz), 124.41 (q, J=32.2 Hz), 123.48 (d, J=0.9 Hz), 119.76 (q, J=4.0 Hz), 117.98, 115.84 (q, J=4.1 Hz). ¹⁹F NMR (376 MHz, DMSO-d₆) δ−60.27, −61.22. LC-MS (ESI): m/z 442.99 [M+H]⁺.

Example 27. 1-(3-Cyanophenyl)-3-(5-(4-(trifluoromethyl)phenoxy)pyrimidin-2-yl) urea (3-py)

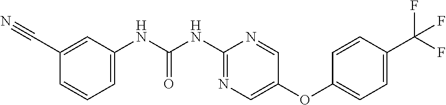 [SMILES: N#Cc1cccc(NC(=O)Nc2ncc(Oc3ccc(C(F)(F)F)cc3)cn2)c1]

The title compound was prepared according to the procedures described in Example 2. White solid (20 mg, 17%); mp 260.2-261.5° C. (EtOAc). ¹H NMR (400 MHz, DMSO-d₆) δ11.52 (s, 1H), 10.49 (s, 1H), 8.66 (s, 2H), 8.09 (s, 1H), 7.91 (d, J =8.0 Hz, 1H), 7.75 (d, J=8.6 Hz, 2H), 7.59-7.41 (m, 2H), 7.29 (d, J=8.5 Hz, 2H). ¹³C NMR (100 MHz, DMSO-d₆) δ160.69 (d, J=0.9 Hz), 154.94, 151.85, 151.26, 145.17, 139.96, 130.71, 128.06 (d, J=3.8 Hz), 126.90, 124.62 (d, J=271.5 Hz), 124.45 (d, J=32.3 Hz), 124.37, 122.53, 119.13, 118.02, 112.15. ¹⁹F NMR (376 MHz, DMSO-d₆) δ−60.28. LC-MS (ESI): m/z 399.99 [M+H]⁺, 441.07 [M+H+41]⁺.

Example 28. 1-(3-Cyano-5-fluorophenyl)-3-(5-(4-(trifluoromethyl) phenoxy)pyrimidin-2-yl)urea (4-py)

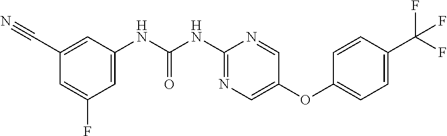 [SMILES: N#Cc1cc(F)cc(NC(=O)Nc2ncc(Oc3ccc(C(F)(F)F)cc3)cn2)c1]

The title compound was prepared according to the procedures described in Example 1. White solid (20 mg, 19%); mp 267.5-270.0° C. (EtOAc). ¹H NMR (400 MHz, DMSO-d₆) δ11.73 (s, 1H), 10.58 (s, 1H), 8.64 (s, 2H), 7.95 (d, J=10.9 Hz, 1H), 7.90 (s, 1H), 7.75 (d, J=6.9 Hz, 2H), 7.48 (d, J=7.4 Hz, 1H), 7.29 (d, J=7.0 Hz, 2H). ¹³C NMR (100 MHz, DMSO-d₆) δ162.38 (d, J=244.4 Hz), 160.58 (d, J=1.8 Hz), 154.73, 151.83, 151.11, 145.39, 141.94 (d, J=12.0 Hz), 128.08 (q, J=3.6 Hz), 124.60 (q, J=271.2 Hz), 124.55 (q, J=32.2 Hz), 119.27 (d, J=3.4 Hz), 118.17, 118.07 (d, J=3.2 Hz), 113.49 (d, J=25.5 Hz), 113.30 (d, J=12.1 Hz), 111.43 (d, J=26.6 Hz). ¹⁹F NMR (376 MHz, DMSO-d₆) δ−60.30, −109.35 (dd, J=11.3, 8.4 Hz). LC-MS (ESI): m/z 418.05 [M+H]⁺, 459.13 [M+H+41]⁺.

Example 29. 1-(5-Fluoro-2-methylphenyl)-3-(5-(4-(trifluoromethyl) phenoxy)pyrimidin-2-yl)urea (7-py)

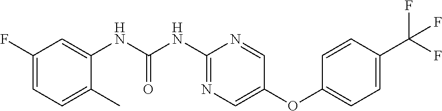 [SMILES: Cc1ccc(F)cc1NC(=O)Nc1ncc(Oc2ccc(C(F)(F)F)cc2)cn1]

The title compound was prepared according to the procedures described in Example 2. White crystalline solid (30 mg, 7%); mp 224.2-226.5° C. (EtOAc). ¹H NMR (400 MHz, DMSO-d₆) δ11.35 (s, 1H), 10.51 (s, 1H), 8.71 (s, 2H), 7.99 (dd, J=11.9, 2.3 Hz, 1H), 7.74 (d, J=8.6 Hz, 2H), 7.27 (d, J=8.5 Hz, 2H), 7.25-7.19 (m, 1H), 6.78 (td, J=8.3, 2.4 Hz, 1H), 2.29 (s, 3H). ¹³C NMR (100 MHz, DMSO-d₆) δ161.01 (d, J=238.4 Hz), 160.91 (d, J=1.3 Hz), 155.15, 151.73, 151.60, 144.87, 138.87 (d, J=11.5 Hz), 131.61 (d, J=9.3 Hz), 128.00 (q, J=4.0 Hz), 124.63 (q, J=271.5 Hz), 124.28 (q, J=32.2 Hz), 122.78 (d, J=2.9 Hz), 117.74, 109.37 (d, J=21.1 Hz), 106.85 (d, J=27.2 Hz), 17.86. ¹⁹F NMR (376 MHz, DMSO-d₆) δ−60.24, −115.62 (m). LC-MS (ESI): m/z 407.14 [M+H]⁺, 448.21 [M+H+41]⁺.

Example 30. 1-(3,4-Difluorophenyl)-3-(5-(4-(trifluoromethyl)phenoxy)pyrimidin-2-yl) urea (10-py)

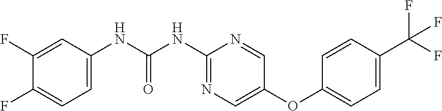 [SMILES: O=C(Nc1ccc(F)c(F)c1)Nc1ncc(Oc2ccc(C(F)(F)F)cc2)cn1]

The title compound was prepared according to the procedures described in Example 2. White solid (40 mg, 39%); mp 224.8-226.2° C. (EtOAc). ¹H NMR (400 MHz, DMSO-d₆) δ11.36 (s, 1H), 10.41 (s, 1H), 8.64 (s, 2H), 7.83-7.77 (m, 1H), 7.75 (d, J=8.9 Hz, 2H), 7.43-7.30 (m, 2H), 7.28 (d, J=8.6 Hz, 2H). ¹³C NMR (100 MHz, DMSO-d₆) δ160.73 (d, J=1.3 Hz), 155.01, 151.80, 151.29, 149.55 (dd, J=243.3, 13.4 Hz), 145.60 (dd, J=241.0, 12.6 Hz), 145.06, 136.09 (dd, J=9.2, 2.9 Hz), 128.03 (q, J=3.7 Hz), 124.61 (d, J=271.6 Hz), 124.41 (d, J=32.3 Hz), 117.96, 117.90 (d, J=17.1 Hz), 116.06 (d, J=2.3 Hz), 108.89 (d, J=21.6 Hz). ¹⁹F NMR (376 MHz, DMSO-d₆) δ−60.30, −137.45 (m), −145.60 (m). LC-MS (ESI): m/z 411.04 [M+H]⁺, 452.12 [M+H+41]⁺.

Example 31. Structure-Activity Relationship (SAR) Studies

All newly synthesized N,N′-disubstituted ureas were initially evaluated by testing in the surrogate eIF2α phosphorylation dual-luciferase reporter (DLR) assay (see e.g., Chen et al, Nat. Chem. Biol. 7 (2011) 610-616; and Ziegeler et al, J. Biol. Chem. 285 (2010) 15408-15419). Briefly, this assay takes advantage of the fact that activated HRI phosphorylates eIF2α which inhibits eIF2 guanine nucleotide exchange factor eIF2B, responsible for exchanging the GDP in the eIF2·GDP complex for GTP. The reduction in the recycling of eIF2·GDP into eIF2·GTP interferes with the formation of eIF2·GTP·Met-tRNAᵢ ternary complex. This results in the inhibition of overall translation, but increases translation of a small subset of mRNAs that contain multiple upstream open reading frames (uORFs) in the 5′ untranslated region (5′UTR). These include mRNA coding for activating transcription factor 4 (ATF-4) (see e.g., Aktas et al, Oncotarget, 4 (2013) 1606-1617; Aktas et al, Oncotarget, 6 (2015) 6902-6914; and Aktas et al, Proc. Natl. Acad. Sci. U.S.A. 95 (1998) 8280-8285).

In the present assay, firefly (F) luciferase mRNA was fused to the 5′UTR of ATF-4 mRNA that had multiple uORFs, while renilla (R) luciferase mRNA was fused to a 5′UTR lacking any uORFs. Agents that reduce the amount of the eIF2·GTP·Met-tRNAᵢ ternary complex, such as N,N′-disubstituted ureas that activate HRI, increase F luciferase expression while reducing the R luciferase expression, resulting in an increased F/R luciferase ratio. The ureas of the present application were tested at 10 μM, 5 μM, 2.5 μM, and 1.25 μM concentrations in 96-well assay plates (see e.g., Chen et al, Nat. Chem. Biol. 7 (2011) 610-616; and Bai et al, ChemBioChem, 14 (2013) 1255-1262). The activity scores as the F/R ratios were calculated for every compound-treated well and normalized to F/R ratio of vehicle-treated (DMSO) wells in the same plate, arbitrarily set at 1 (F/R=1). Data obtained in the surrogate dual-luciferase eIF2α phosphorylation (DLR) assay for the series of 1-phenyl-3/4-(4-trifluorometyl) phenoxy-cycloalkyl)ureas (4-CF₃-ΦcAlkΦUs) and 1-phenyl-3-(5-(4-(trifluoromethyl) phenoxy)pyrimidin-2-yl)ureas (4-CF₃-ΦOPyΦUs) described herein are shown in Tables 1-2.

TABLE 1

| Substituents on | | | | Fold stimulation of ATF-4 Surrogate eIF2α @ | | | | | | | | | cLogP | | |
|---|---|---|---|---|---|---|---|---|---|---|---|---|---|---|---|
| the N-phenyl | | | | 1.25 μM | | | 5 μM | | | IC₅₀^{a} [μM] | | | | c- | c- |
| R₁ | R₂ | R₃ | R₄ | c-Hex^{b} | c-Pent^{c} | c-But^{d} | c-Hex^{b} | c-Pent^{c} | c-But^{d} | c-Hex^{b} | c-Pent^{c} | c-But^{d} | c-Hex^{b} | Pent^{c} | But^{d} |
| H | CF₃ | H | CN | 5.7 ± 1.4 | 3.1 ± 0.7 | 4.4 ± 1.1 | 13.8 ± 1.5 | 8.3 ± 2.4 | 10 ± 1.8 | 0.35 ± 0.1 | 1.2 ± 0.5 | 0.9 ± 0.3 | 6.20 | 6.08 | 5.96 |
| | | | | 1-VI | 1-V | 1-IV | 1-VI | 1-V | 1-IV | 1-VI | 1-V | 1-IV | 1-VI | 1-V | 1-IV |
| | | | | | | 4.3 ± 0.6 | | | 8.4 ± 0.5 | | | 0.9 ± 0.3 | | | 5.96 |
| | | | | | | 1-IVc | | | 1-IVc | | | 1-IVc | | | 1-IVc |
| H | CF₃ | H | H | 3.6 ± 1.2 | 1.2 ± 0.4 | 2.4 ± 0.6 | 9.2 ± 1.4 | 7 ± 0.9 | 6.5 ± 1.2 | 0.9 ± 0.5 | 2.5 ± 0.5 | 3.4 ± 1 | 6.41 | 6.29 | 6.16 |
| | | | | 2-VI | 2-V | 2-IV | 2-VI | 2-V | 2-IV | 2-VI | 2-V | 2-IV | 2-VI | 2-V | 2-IV |
| | | | | | | 1.2 ± 0.3 | | | 6.2 ± 1.2 | | | 2.6 ± 0.4 | | | 6.15 |
| | | | | | | 2-IVc | | | 2-IVc | | | 2-IVc | | | 2-IVc |
| H | CN | H | H | 1.9 ± 0.4 | 0.8 ± 0.2 | 1.6 ± 0.2 | 6.1 ± 0.9 | 1.6 ± 0.2 | 3.5 ± 0.4 | 1.6 ± 1 | 6.5 ± 2.3 | 4.9 ± 0.9 | 5.13 | 5.01 | 4.78 |
| | | | | 3-VI | 3-V | 3-IV | 3-VI | 3-V | 3-IV | 3-VI | 3-V | 3-IV | 3-VI | 3-V | 3-IV |
| | | | | | | 1.1 ± 0.2 | | | 0.9 ± 0.1 | | | >10 | | | 4.78 |
| | | | | | | 3-IVc | | | 3-IVc | | | 3-IVc | | | 3-IVc |
| H | F | H | CN | 4.6 ± 0.7 | 2.4 ± 0.3 | 2 ± 0.4 | 9.3 ± 1.3 | 8 ± 2 | 5.3 ± 0.6 | 0.63 ± 0.3 | 1.9 ± 1.3 | 2.8 ± 1.3 | 5.38 | 5.26 | 5.03 |
| | | | | 4-VI | 4-V | 4-IV | 4-VI | 4-V | 4-IV | 4-VI | 4-V | 4-IV | 4-VI | 4-V | 4-IV |
| H | F | CN | H | 5.5 ± 0.4 | | | 10.4 ± 1 | | | 0.46 ± 0.1 | | | 5.38 | | |
| | | | | 5-VI | | | 5-VI | | | 5-VI | | | 5-VI | | |
| F | H | H | CN | 1 ± 0.3 | | | 1.5 ± 0.5 | | | >10 | | | 4.93 | | |
| | | | | 6-VI | | | 6-VI | | | 6-VI | | | 6-VI | | |
| CH₃ | H | H | F | 1.8 ± 0.8 | 1.4 ± 0.1 | 0.7 ± 0.2 | 1.5 ± 0.4 | 1.3 ± 0.4 | 1.4 ± 0.1 | 5.3 ± 1.8 | 8 ± 2.5 | >10 | 5.38 | 5.26 | 5.03 |
| | | | | 7-VI | 7-V | 7-IV | 7-VI | 7-V | 7-IV | 7-VI | 7-V | 7-IV | 7-VI | 7-V | 7-IV |
| F | H | CN | H | 2.4 ± 0.4 | | | 6.2 ± 1.3 | | | 1.4 ± 0.3 | | | 4.93 | | |
| | | | | 8-VI | | | 8-VI | | | 8-VI | | | 8-VI | | |
| H | CN | F | H | 3.7 ± 0.8 | | | 6.1 ± 1.1 | | | 1.4 ± 0.3 | | | 5.38 | | |
| | | | | 9-VI | | | 9-VI | | | 9-VI | | | 9-VI | | |
| H | F | F | H | 2.9 ± 0.7 | 0.9 ± 0.2 | 2.2 ± 0.5 | 5.6 ± 1.2 | 3.5 ± 0.7 | 4.5 ± 0.4 | 1.3 ± 0.3 | 5.2 ± 1.5 | 5.5 ± 0.7 | 5.62 | 5.50 | 5.27 |
| | | | | 10-VI | 10-V | 10-IV | 10-VI | 10-V | 10-IV | 10-VI | 10-V | 10-IV | 10-VI | 10-V | 10-IV |
| F | F | H | H | 4.7 ± 1 | | 0.8 ± 0.1 | 9.9 ± 1.3 | | 6.2 ± 2.7 | 1.6 ± 02 | | 2.3 ± 0.9 | 5.17 | | 4.82 |
| | | | | 11-VI | | 11-IV | 11-VI | | 11-IV | 11-VI | | 11-IV | 11-VI | | 11-IV |

^{a}Concentration that inhibits growth of human melanoma CRL-2813 cells bry 50%;
^{b}c-Hex (cyclohexyl, m = 1, n = 1);
^{c}c-Pent (cyclopentl, m = 0, n = 1);
^{d}c-But (cyclobutyl, m = 0, n = 0)

TABLE 2
Substitution Pattern and cLogP of the 1-phenyl-3-(5-(4-
(trifluoromethyl)phenoxy-pyrimidin-2-yl)ureas
((4-CF₃)ΦOPyrΦUs)*

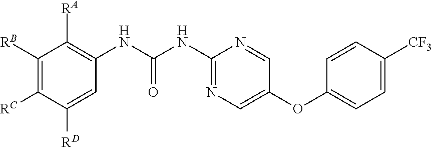

| | R^{A} | R^{B} | R^{C} | R^{D} | cLogP |
|---|---|---|---|---|---|
| 1-py | H | CF₃ | H | CN | 5.27 |
| 2-py | H | CF₃ | H | H | 5.80 |
| 3-py | H | CN | H | H | 4.37 |
| 4-py | H | F | H | CN | 4.52 |
| 7-py | CH₃ | H | H | F | 4.97 |
| 10-py | H | F | F | H | 5.12 |

*No activity in the surrogate dual-luciferase eIF2α phosphorylation assay up to 20 μM.

Previous reports have described two 1-phenyl-3-((1,4-trans)-4-(4-trifluorometyl) phenoxy)cyclohexyl)ureas (4-CF₃-ΦOcHΦUs), 1-VI and 2-VI (see e.g., Chen et al, J. Med. Chem. 56 (2013) 9457-9470; and Yefidoff-Freedman et al, J. Med. Chem. 60 (2017) 5392-5406). Both compounds have a (4-trifluoromethy) phenoxy ring and while 2-VI contains a N-(3-trifluoromethyl)phenyl, compound 1-VI contains a N-(3-trifluormethyl-5-cyano)phenyl moiety, leading to slightly different cLogP (6.41 and 6.20, respectively). Both compounds activate HRI, and 1-VI was more active (3.6- and 5.7-fold increase in the surrogate eIF2α phosphorylation assay at 1.25 μM for 2-VI and 1-VI, respectively).

The 4-CF₃-ΦOcHΦUs described herein were prepared to reduce hydrophobicity without compromising potency. The compounds provided herein contain a (4-trifluoromethyl)phenoxy moiety and differ in nature and position of the substituents on the N-phenyl moiety compared to compounds 2-VI and 1-VI. The most potent analog in this library, compound 5-VI, was almost equipotent with 1-VI (5.5- and 5.7-fold increase at 1.25 μM, respectively) but exhibited significantly lower cLogP than the latter (cLogP=5.38 vs 6.20, respectively). Moreover, the 2,3-difluorophenyl analog, 4-CF₃-ΦOcHΦU 11-VI, exhibited potency that falls between 1-VI and 2-VI, but is somewhat less hydrophobic than 5-VI (cLogP=5.17 vs 5.38, respectively). Without being bound by theory, it is believed that replacement of the hydrophobic CF₃ substituent in 1-VI with F, a smaller and less hydrophobic substituent (cf. 1-VI with 5-VI) resulted in an insignificant loss in potency but a significant reduction in hydrophobicity. The least hydrophobic 4-CF₃-ΦOcHΦUs in this series, compounds 6-VI and 8-VI, presenting two polar and electron withdrawing substituents as either N-(2-fluoro-5-cyano)phenyl or N-(2-fluoro-4-cyano)phenyl, respectively, were not among the most potent analogs. Without being bound by theory, it is believed that the nature of substituents and the positions on the N-phenyl ring are more important for high potency than the overall hydrophobicity.

The 4-CF₃-ΦOcBΦUs series described herein contains three pairs of cis and trans isomers, compounds 1-IV and 1-IVc, 2-IV and 2-IVc, and 3-IV and 3-IVc, listed in the decreasing order of potency but almost equipotent within the pairs (e.g., causing 4.4- and 4.3-fold increase in the F/R ratio in the surrogate eIF2α phosphorylation assay at 1.25 μM for 1-IV and 1-IVc, respectively, and IC₅₀ of 0.9 μM for both vs increasing F/R ratio 1.6- and 1.1-fold at 1.25 μM and IC₅₀=4.9 and >10 μM for 3-IV and 3-IVc, respectively; see Table 1). In view of the greater commercial availability of (1,4-trans)-4-aminocyclohexan-1-ol and its greater metabolic stability (see e.g., Shen et al, J. Med. Chem. 55 (2012) 1789-1808) the trans isomer was selected for further development.

Without being bound by theory, it is believed that the cyclohexyl moiety in the ΦOcHΦU series serves as a scaffold to link between the two parts of the pharmacophore, the N-phenyl substituted urea moiety and the substituted phenoxy moiety. As such, reducing the size of the cycloalkyl linker would reduce the overall hydrophobicity and also affect the overall rigidity of the N-aryl urea. Accordingly, two libraries of 1-phenyl-3-((1,3-trans)-4-(4-trifluorometyl) phenoxy)cyclopentyl)ureas (4-CF₃-ΦOcPΦUs) and 1-phenyl-3-(( 1,3-trans )-4-(4-trifluorometyl)phenoxy)cyclobutyl)pureas (4-CF₃-ΦOcBΦUs) were prepared and are described herein. It was found that reducing the ring size of the cycloalkyl moiety was accompanied by a consistent lowering of cLogP (e.g. 6.41, 6.29, 6.16 for the 4-CF₃-ΦOcHΦU-2-VI, 4-CF₃-ΦOcPΦU-2-V and 4-CF₃-ΦOcBΦU-2-IV, respectively). None of the analogs incorporating either the cyclobutyl or the cyclopentyl moieties were more potent or even equipotent to the corresponding cyclohexyl containing analog. Possibly, the conformational rigidification accompanying reduction in ring size and/or compromise of hydrophobic interactions enabled by the cyclohexyl ring prevented both the 4-CF₃-ΦOcPΦUs and the 4-CF₃-ΦOcBΦUs from achieving the same effective target complementing interactions provided by the 4-CF₃-ΦOcHOUs.

Replacement of the (1,4-trans)-4-(4-trifluoromethyl)phenoxycyclohexyl moiety in 4-CF₃-ΦOcHΦUs with 5-(4-trifluoromethyl)phenoxy)pyrimidin-2-yl as in 4-CF₃-ΦOPyΦUs was also conducted to test a different mode of reducing hydrophobicity by replacing the 1,4-disubstituted cyclohexyl with 2,5-disubstituted pyrimid-2-yl (cf. e.g. cLogP of 2-VI and 2-py 6.41 and 5.80, respectively), and also testing for a mode of global molecular rigidification. This rigidification involved the replacement of the flexible puckering cyclohexyl ring with the aromatic planar pyrimidyl ring and enabled the formation of a planar conjugated pseudo six-membered ring generated by an intramolecular hydrogen bond within the pyrimidin-2-yl-urea motif, as shown in Scheme 3), which was calculated to be more stable than the extended conformation (see e.g., Furet et al, Bioorg. Med. Chem. Lett. 18 (2008) 897-900; and Guagnano et al, J. Med. Chem. 54 (2011) 7066-7083). Due to the pairing of one hydrogen bond donor and one hydrogen bond acceptor to pseudo six-membered ring, these H-donor and -acceptors are no longer available for intermolecular interactions with water and therefore aqueous solubility was lower than the one anticipated from compounds of the same cLogP that do not form intramolecular hydrogen bonds (see e.g., Kuhn et al, J. Med. Chem. 53 (2010) 2601-2611). All 4-CF₃-ΦOPyΦUs compounds described herein were devoid of activity in the surrogate eIF2α phosphorylation assay up to 20 μM as measured in the DLR assay. Without being bound by theory, it is believed that this mode of molecular rigidification may lock compounds in an orientation that cannot accommodate the HRI activation and therefore did not lead to the activity in the surrogate eIF2α phosphorylation assay, as shown below in Scheme 3.

Scheme 3. Potential Molecular Rigidification by a Putative Planar Pseudo Six-Membered Ring Generated by an Intramolecular Hydrogen Bond within the Pyrimidin-2-yl-urea Motif

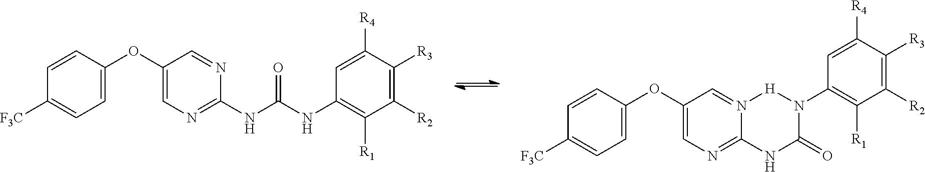

Example 32. Biological Activities in Secondary Mechanistic Assays

To validate the 4-CF₃-ΦOcAlkΦUs and 4-CF₃-ΦOPyΦUs described herein as activators of HRI, and thereby inducers of eIF2α phosphorylation, representative 4-CF₃-ΦOcHΦUs (4-VI, 5-VI, 6-VI, 7-VI, 11-VI and 1-VI) and 4-CF₃-ΦOcBΦU (1-IV) (see Table 1) were tested in secondary mechanistic assays that included phosphorylation of endogenous eIF2α and expression of its downstream effector CHOP.

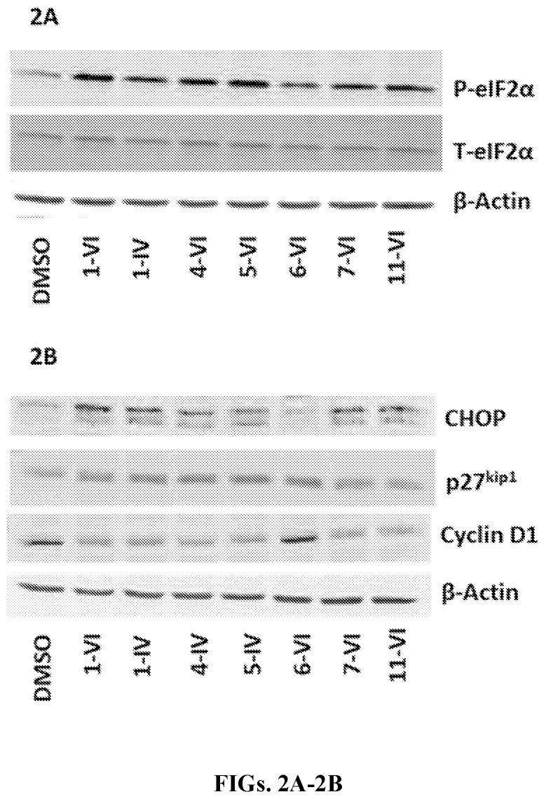

Endogenous eIF2α is the best-known substrate of HRI and the upstream regulator of the eIF2·GTP·Met-tRNAᵢ ternary complex abundance, while CHOP expression is a downstream effector of eIF2α phosphorylation. For assessing eIF2α phosphorylation, blotted cell lysates were treated for two hours with vehicle or selected compounds using antibodies specific for the total-eIF2α and the phosphorylated-eIF2α (T-eIF2α and [PhoS⁵¹]-eIF2α), respectively (see e.g., Aktas et al, J. Nutr. 134 (2004) 2487S-2491S) (see FIG. 2A). The least potent analog, 6-VI, in stimulating eIF2α phosphorylation as measured by the surrogate eIF2α phosphorylation assay was also the least active in phosphorylating endogenous eIF2α in adherent human melanoma CRL-2813 cells as determined by Western blot analysis. Moreover, in CRL-2813 cells treated with the N,N′-disubstituted ureas for eight hours, expression of CHOP protein as well as expression of cell cycle regulatory proteins, the oncogenic protein cyclin D1 and the cyclin dependent kinase (CDK) inhibitor p27^{KiP1} that prevents activation of cyclin/CDK complexed at the G1 phase of cell cycle, was revealing. Relative to DMSO, the control vehicle, 4-CF₃-ΦOcHΦUs (1-VI, 4-VI, 5-VI, 7-VI and 11-VI), and 4-CF₃-ΦOcBΦUs (1-IV) increased the expression of CHOP, a downstream effector of eIF2α phosphorylation, and decreased the expression of cyclin D1 whose expression is dependent on the abundance of the ternary complex, as shown in FIG. 2B (see e.g., Aktas et al, Proc. Natl. Acad. Sci. U.S.A. 95 (1998) 8280-8285; and Bai et al, ChemBioChem, 14 (2013) 1255-1262). 4-CF₃-ΦOcHΦU 6-VI, which was inactive in the DLR assay, did not increase expression of CHOP and did not inhibit the expression of cyclin D1, as shown in FIG. 2B (see e.g., Aktas et al, Proc. Natl. Acad. Sci. U.S.A. 95 (1998) 8280-8285; and Aktas et al, Mol. Cell. Biol. 17 (1997) 3850-3857). None of the analogs affected the expression of housekeeping proteins such as p27^{KiP1} and β-actin, as shown in FIG. 2B).

In summary, the results obtained from the secondary and tertiary mechanistic assays reported herein agreed with the data from the surrogate eIF2α phosphorylation reporter assay and supported the conclusion that the active 4-CF₃-ΦOcAlkΦUs were targeting the ternary complex by activating HRI.

Example 33. In Vitro Cell Proliferation Studies

All 4-CF₃-ΦOcAlkΦUs were tested in the sulforhodamine B (SRB) cell proliferation assay to establish the concentrations that inhibit human melanoma CRL-2813 cells growth by 50% (IC₅₀) (see Table 1). This activity reflects a combined effect of membrane permeability and overall anti-proliferative effects that include on target and off-target effects. The most active analogs that are part of the 4-CF₃-ΦOcAlkylΦU series displayed IC₅₀ in the sub-micromolar range (e.g., 0.46 and 0.9 μM for 5-VI and 1-IV, respectively). There was also good correlation between the activity of the compounds in the surrogate eIF2α phosphorylation reporter assay and the IC₅₀ values in the cell proliferation assay (e.g., 5.7-, 3.1- and 4.4-fold at 1.25 μM and 13.8-, 8.3- and 10-fold at 5 μM in the surrogate eIF2α-P assay vs IC_{50S} of 0.35, 1.2 and 0.9 μM, for 1-VI, 1-V, and 1-IV, respectively; see Table 1). Together, these results support the hypothesis that anti-proliferative activity of 4-CF₃-ΦOcAlkylΦUs analogs is primarily contributed through the activation of HRI. These results are also consistent previously reports of 4-CF₃-ΦOcHΦUs 1-VI, 2-VI, and 3-VI, which inhibit cell proliferation by activating HRI and inducing eIF2α phosphorylation (see e.g., Chen et al, Nat. Chem. Biol. 7 (2011) 610-616; Denoyelle et al, Bioorg. Med. Chem. Lett. 22 (2012) 402-409; Chen et al, J. Med. Chem. 56 (2013) 9457-9470; and Yefidoff-Freedman et al, J. Med. Chem. 60 (2017) 5392-5406).

Example 34. Specificity of HRI

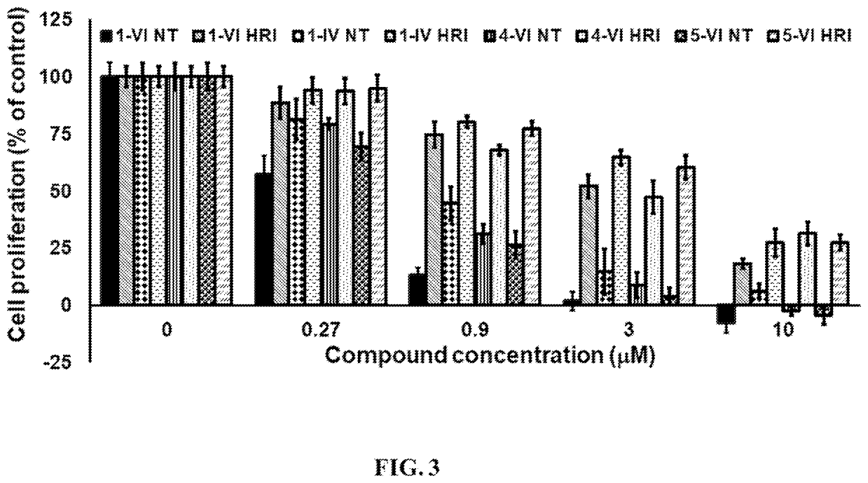

To determine the specificity of the compounds described herein for HRI, knockdown expression of HRI in MCF-7 cells using a pool of siRNA targeting HRI was performed, along with a cell proliferation assay as a biologic response parameter (see e.g., Chen et al, Nat. Chem. Biol. 7 (2011) 610-616; and Chen et al, J. Med. Chem. 56 (2013) 9457-9470). This assay was selected because it compares specificity of the compounds for HRI compared to all other cellular targets that can impinge on cell proliferation. MCF-7 human breast cancer cells transfected with siRNA targeting HRI or vehicle were treated with various concentrations of representative 4-CF₃-ΦOcHΦUs (1-VI, 4-VI, and 5-VI) and 4-CF₃-ΦOcBΦU (1-IV). MCF-7 cells were selected because knockdown efficiency in these cells is significantly higher than in CRL-2813 cells (see e.g., Chen et al, Nat. Chem. Biol. 7 (2011) 610-616). As shown in FIG. 3 knocking down HRI caused a dramatic reduction in the activity of all four compounds tested. These data demonstrate that the tested compounds specifically activate HRI.

Example 35. Data Analysis

One objective of the experiments described herein was to provide new compounds with lower lipophilicity and, if possible, enhanced potency compared to previously reported compounds 1-VI and 2-VI. As described herein, replacing the CF₃ substituent from the N-phenyl in 1-VI and replacing it with F generating N-(3-F,5-CN) phenyl and N-(3-F,4-CN)phenyl moieties as in 4-VI and 5-VI, respectively, resulted in compounds that were as potent as compounds 1-VI and 2-VI but exhibited significantly lower lipophilicity as estimated from their cLogP both 5.23 (cf. 6.20 for 1-VI). It was also found that reducing lipophilicity by replacing the (1,4-trans)-disubstituted cyclohexyl ring with smaller cycloalkyls such as cyclobutyl and cyclopentyl resulted in lower potency in the DLR assay. Replacement of the (1,4-trans)-disubstituted cyclohexyl ring with a 2,5-disubstituted pyrimidine ring resulted in significantly lower cLogPs but reduced apparent solubility and abolished potency as measured in the surrogate eIF2α phosphorylation assay. The planarity and aromatic nature of the pyrimidine ring and the putative extended rigidification originating from the potential formation of an intramolecular hydrogen bond within the pyrimidin-2-yl-urea motif do not accommodate interactions with the solvent or productive interactions with the cognate macromolecular targets of these ligands. Taken together, these results underscore a unique role of the (1,4-trans)-disubstituted cyclohexyl moiety as an advantageous linker connecting the two pharmacophore fragments, the N-phenyl-ureido and the phenoxy motif, allowing the pharmacophore fragments to acquire the correct spatial orientation by providing the right molecular flexibility, bulk, and lipophilicity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

1-43. (canceled)

44. A compound of Formula I: 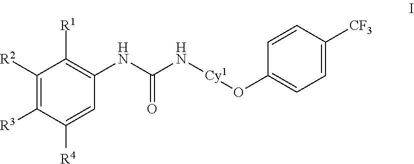
or a pharmaceutically acceptable salt thereof, wherein:
Cy¹ is selected from the group consisting of a C₃₋₁₀ cycloalkyl ring and a 5-10 membered heteroaryl ring;
R¹, R², R³, and R⁴ are each independently selected from the group consisting of H, halo, C₁₋₆ alkyl, C₁₋₆ haloalkyl, and cyano, wherein at least one of R¹, R², R³, and R⁴ is not H;
provided that the compound of Formula I is not selected from the group consisting of:

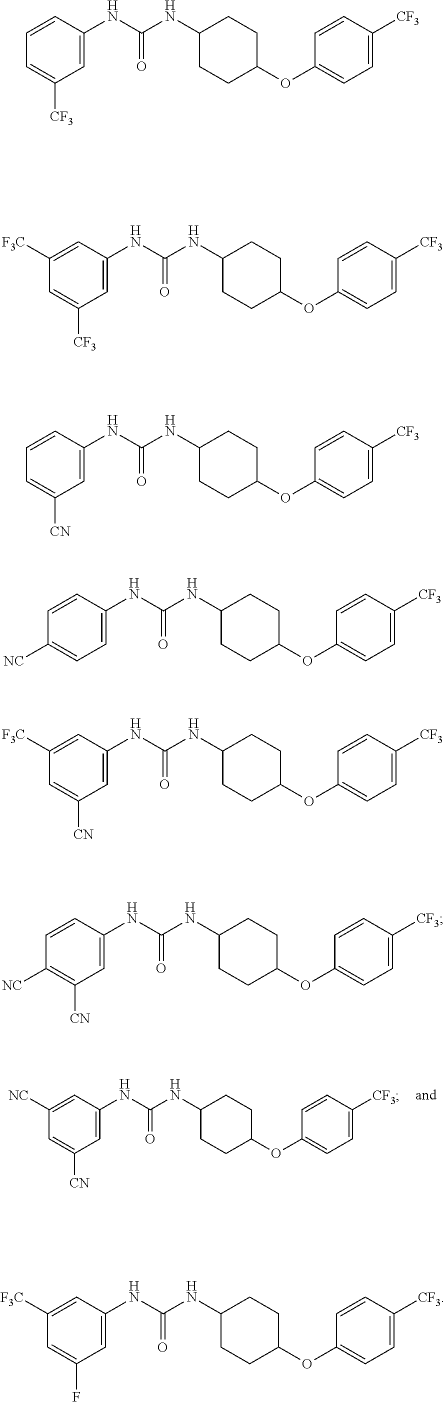

45. The compound of claim 44, or a pharmaceutically acceptable salt thereof, wherein Cy¹ is cyclohex-1,4-diyl.

46. The compound of claim 44, or a pharmaceutically acceptable salt thereof, wherein Cy¹ is selected from cyclobut-1,3-diyl and cyclopent-1,3-diyl.

47. The compound of claim 44, or a pharmaceutically acceptable salt thereof, wherein Cy¹ is a 5-6 membered heteroaryl ring.

48. The compound of claim 47, or a pharmaceutically acceptable salt thereof, wherein Cy¹ is pyrimidinyl.

49. The compound of claim 44, or a pharmaceutically acceptable salt thereof, wherein:
R¹ is selected from the group consisting of H, halo, and C₁₋₃ alkyl;
R² is selected from the group consisting of H, halo, C₁₋₃ alkyl, C₁₋₃ haloalkyl, and cyano;
R³ is selected from the group consisting of H, halo, and cyano; and
R⁴ is selected from the group consisting of H, halo, and cyano.

50. The compound of claim 44, or a pharmaceutically acceptable salt thereof, wherein:
R¹ is selected from the group consisting of H, fluoro, and C₁₋₃ alkyl;
R² is selected from the group consisting of H, fluoro, C₁₋₃ alkyl, C₁₋₃ haloalkyl, and cyano;
R³ is selected from the group consisting of H, fluoro, and cyano; and
R⁴ is selected from the group consisting of H, fluoro, and cyano.

51. The compound of claim 44, wherein the compound of Formula I is a compound of Formula II: 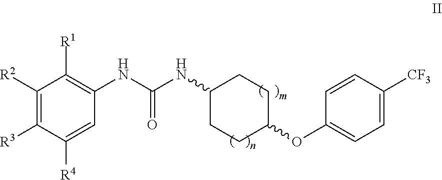
or a pharmaceutically acceptable salt thereof, wherein n and m are each independently 0, 1, 2, or 3.

52. The compound of claim 51, wherein m is 0 and n is 1.

53. The compound of claim 51, wherein m is 0 and n is 0.

54. The compound of claim 51, wherein m is 1 and n is 1.

55. The compound of claim 44, wherein the compound of Formula I is a compound of Formula III: 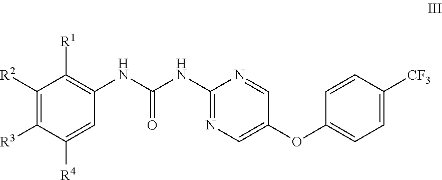
or a pharmaceutically acceptable salt thereof.

56. The compound of claim 1 selected from any one of the following compounds: 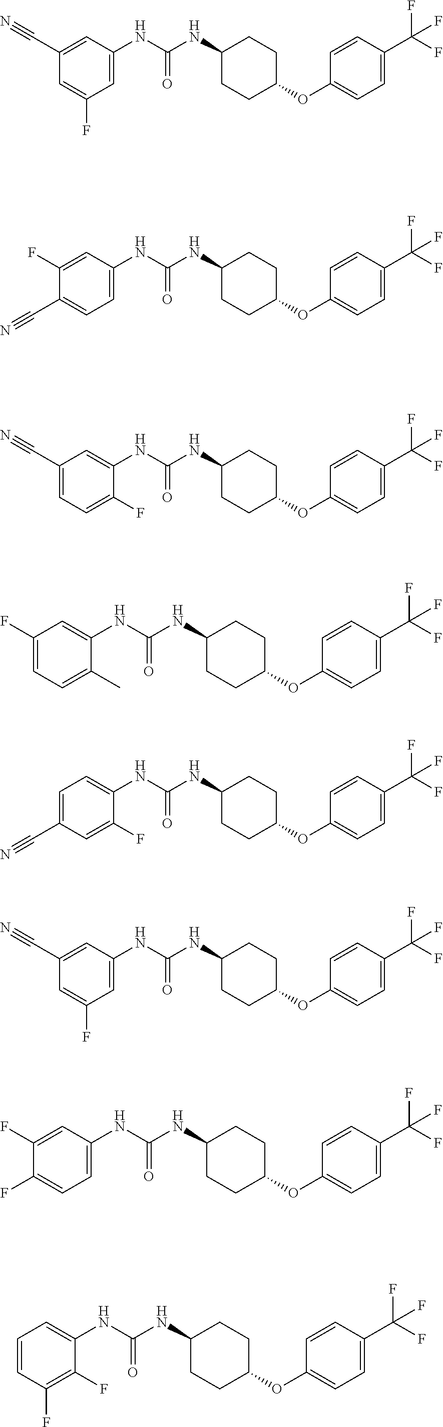
or a pharmaceutically acceptable salt thereof.

57. The compound of claim 44 selected from any one of the following compounds: 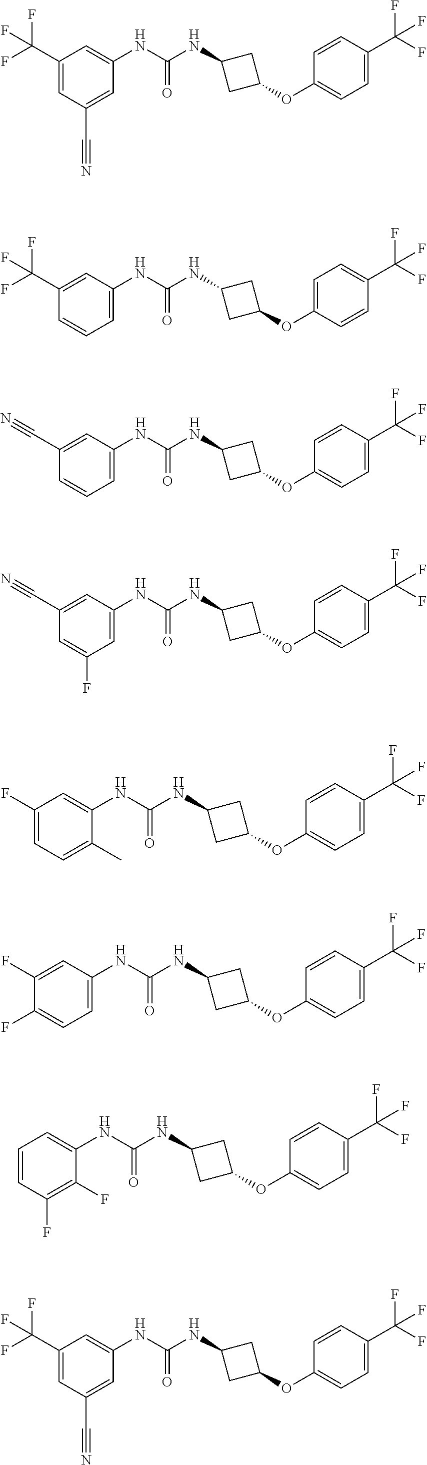 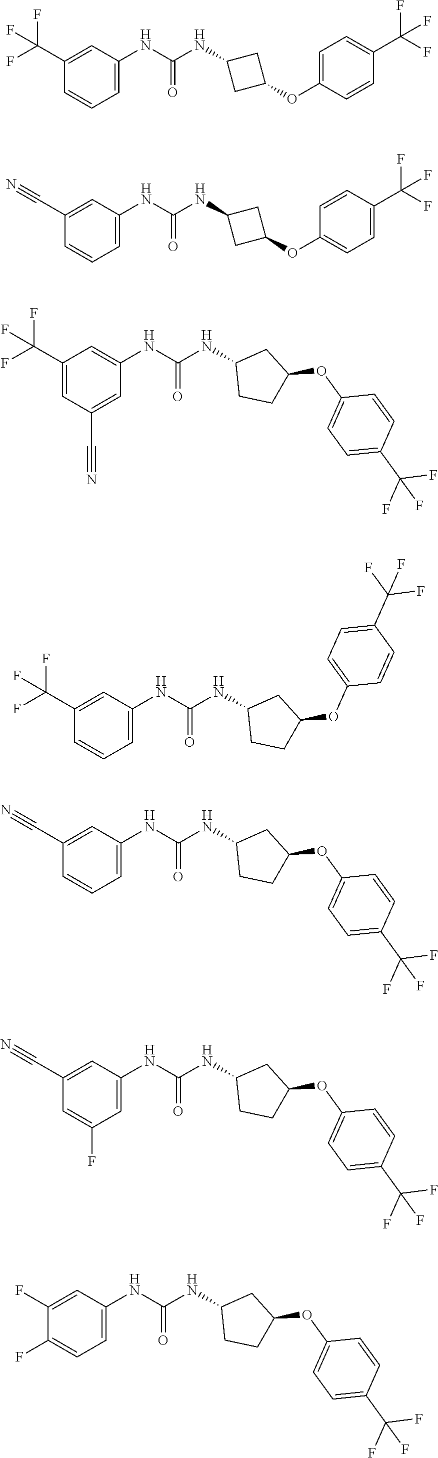 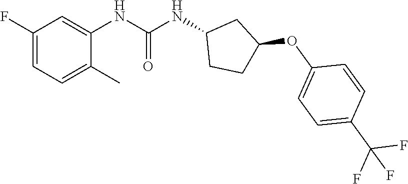 [SMILES: Cc1ccc(F)cc1NC(=O)N[C@H]1CC[C@H](Oc2ccc(C(F)(F)F)cc2)C1]
or a pharmaceutically acceptable salt thereof.

58. The compound of claim 44 selected from any one of the following compounds: 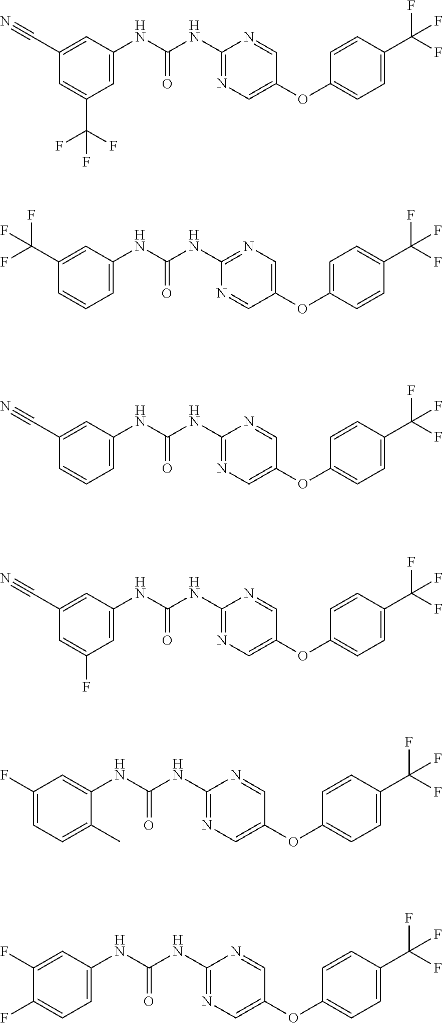 [SMILES: N#Cc1cc(NC(=O)Nc2ncc(Oc3ccc(C(F)(F)F)cc3)cn2)cc(C(F)(F)F)c1 ; O=C(Nc1cccc(C(F)(F)F)c1)Nc1ncc(Oc2ccc(C(F)(F)F)cc2)cn1 ; N#Cc1cccc(NC(=O)Nc2ncc(Oc3ccc(C(F)(F)F)cc3)cn2)c1 ; N#Cc1cc(F)cc(NC(=O)Nc2ncc(Oc3ccc(C(F)(F)F)cc3)cn2)c1 ; Cc1ccc(F)cc1NC(=O)Nc1ncc(Oc2ccc(C(F)(F)F)cc2)cn1 ; O=C(Nc1ccc(F)c(F)c1)Nc1ncc(Oc2ccc(C(F)(F)F)cc2)cn1]
or a pharmaceutically acceptable salt thereof.

59. A pharmaceutical composition comprising a compound of claim 44, or a pharmaceutically acceptable salt thereof, and a pharmaceutically acceptable excipient.

60. A method of activating one or more eIF2α kinases in a cell, the method comprising contacting the cell with an effective amount of a compound of claim 44, or a pharmaceutically acceptable salt thereof.

61. A method of treating a disease or disorder associated with abnormal activity or expression of one or more eIF2α kinases in a patient, the method comprising administering to the patient a therapeutically effective amount of a compound of claim 44, or a pharmaceutically acceptable salt thereof.

62. The method of claim 61, wherein the disease or disorder is a cancer selected from cervical cancer, liver cancer, bile duct cancer, eye cancer, esophageal cancer, head and neck cancer, brain cancer, prostate cancer, pancreatic cancer, skin cancer, testicular cancer, breast cancer, uterine cancer, penile cancer, small intestine cancer, colon cancer, stomach cancer, bladder cancer, anal cancer, lung cancer, lymphoma, leukemia, thyroid cancer, bone cancer, kidney cancer, ovarian cancer, and multiple myeloma.

63. The method of claim 61, wherein the disease or disorder is selected from hemolytic anemia not caused by an infectious agent, Wolcott-Rallison syndrome, neurodegenerative or motor neuron disease, diabetes, non-alcoholic fatty liver disease, tuberous sclerosis complex, an autism spectrum disorder, a ribosomal defect disease, and a mental retardation disorder.
